(19) United States
(12) Patent Application Publication
(10) Pub. No.: US 2003/0159164 A1
(43) Pub. Date: Aug. 21, 2003

(54) Compositions and methods for the synthesis of fatty acids, their derivatives and downstream products

(76) Inventors: John Joseph Kopchick, Athens, OH (US); Bruce Kelder, Athens, OH (US); Yung-Sheng Huang, Arlington, OH (US); Stephen J. Kirchner, Westerville, OH (US); Pradip Mukerji, Gahanna, OH (US)

Correspondence Address: MEDLEN & CARROLL, LLP, Suite 350, 101 Howard Street, San Francisco, CA, 94105, US

(21) Appl. No.: 10/278,391

(22) Filed: Oct. 23, 2002

Related U.S. Application Data
(63) Continuation of application No. 09/087,578, filed on May 29, 1998, now abandoned.

(51) Int. Cl. A01K 67/027; C12P 7/64; C12N 9/04; C12P 21/02; C12N 5/06

(52) U.S. Cl. 800/14; 800/15; 800/16; 800/17; 800/18; 435/134; 435/190; 435/320.1; 435/325; 426/583

(57) ABSTRACT

The invention generally relates to synthesis of essential fatty acids and their derivatives, long-chain polyunsaturated fatty acids (LC-PUFAs) and eicosanoids in transfected cells and in transgenic animals.

### FIELD OF THE INVENTION

The invention generally relates to compositions and methods for the synthesis of essential fatty acids, their derivatives and downstream products, as well as altered levels of long-chain polyunsaturated fatty acids (LC-PUFAs) and eicosanoids in transfected cultured mammalian cells and in transgenic animals.

### BACKGROUND OF THE INVENTION

Both animal and plants have the ability to synthesize fatty acids with chain lengths up to 18-carbons and to desaturate fatty acids at the 9 position. However, during the course of evolution, animals have lost the ability to insert double bonds into fatty acids beyond the 9 position, for example, to insert double bonds into 12 and 15 positions. As a result, animals cannot convert oleic acid (18:1n9) to linoleic acid (18:2n6) and linoleic acid to α-linolenic acid (18:3n3).

Linoleic and α-linolenic acids and their derivatives are required by animals to maintain their normal physiological functions. They, as vitamins, must be taken in via the diet. To be fully useful, these fatty acids need to be metabolized. In mammalians, these two acids can be metabolized by common enzyme systems. They are first converted to γ-linolenic acid (GLA, 18:3n6) and stearidonic acid (SDA, 18:4n3), respectively, by the action of delta 6-desaturase. They are then elongated by elongase to form dihomo-γ-linolenic acid (DGLA, 20:3n6) and (n3) eicosatetraenoic acid (20:4n3), respectively, and further metabolized by delta 5-desaturase to form arachidonic acid (AA, 20:4n6) and eicosapentaenoic acid (EPA, 20:5n3), respectively. Both AA and EPA can be further metabolized to other long-chain polyunsaturated fatty acids (LC-PUFAs) [See FIG. 1]. LC-PUFAs are major constituents in cell membranes. DGLA, AA and EPA can also serve as precursors of 1, 2, and 3-series prostaglandin, thromboxanes and leukotriene biosynthesis, respectively, and these eicosanoids regulate a wide range of physiological functions. Adequate supply of these precursors is also vitally important for maintaining normal physiological activities.

Since 6-desaturation is considered to be the rate-limiting step in the synthesis of LC-PUFAs, the essential ability of animals to convert these acids to LC-PUFA is paramount. The need of LC-PUFAs is further enhanced during the period of rapid growth. There are reports that infants whose diets are not provided with LC-PUFAs, particularly AA and docosahexaenoic acid (DHA, 22:6n3) (which are derivatives of linolenic acid) display significant differences both in biochemical parameters and also in functional properties, such as visual acuity and psychomotor tests in comparison with breast-fed infants. Normally, an infant receives these acids with the mother's milk, because human milk contains both n6 and n3 LC-PUFAs. Most of infant formulas on the market do not contain LC-PUFAs. Attempts to simulate the fat mixture in human milk on the basis of animal, vegetable and microbial oils or fats has often been encountered with the very costly raw materials, which in many cases are still not available in sufficient quantity to meet the need (See U.S. Pat. No. 5,709,888 incorporated herein by reference). Hence, what is required are alternative sources of EFA and LC-PUFA, that are commonly used in commodities, such as milk, infant formula, dietary supplement and pharmaceuticals.

### SUMMARY OF THE INVENTION

The invention generally relates to compositions and methods for the synthesis of essential fatty acids, their derivatives and downstream products, as well as altered levels of long-chain polyunsaturated fatty acids (LC-PUFAs) and eicosanoids in transfected cells and in transgenic animals. In one embodiment, the present invention contemplates introducing nucleic acid encoding a heterologous desaturase gene into an animal cell under conditions such that said cells synthesize essential fatty acids or demonstrate altered levels of long-chain polyunsaturated fatty acids. In another embodiment, the present invention contemplates introducing nucleic acid encoding a heterologous regulatory element in operable combination with either a heterologous or a homologous desaturase gene into an animal cell under conditions such that said cells display altered levels of long-chain polyunsaturated fatty acid synthesis and/or synthesize essential fatty acids.

In a specific embodiment, the present invention contemplates introducing nucleic acid encoding a non-mammalian desaturase gene into a mammalian cell under conditions such that said cells synthesize essential fatty acids and/or display altered levels of long-chain polyunsaturated fatty acids. Thus, the present invention contemplates vectors comprising nucleic acid encoding a desaturase gene, said vector capable of transfecting mammalian cells. Moreover, the present invention contemplates mammalian cells (including cells in tissue culture and in bioreactors), as a composition, which synthesize essential fatty acids (e.g., linoleic and/or linolenic fatty acids).

The present invention also contemplates human and non-human transgenic animals comprising heterologous desaturase genes, as well as human and non-human transgenic animals comprising heterologous regulatory elements in operable combination with either heterologous or homologous desaturase genes. In particular, the present invention contemplates vectors comprising a tissue-specific promoter in operable combination with nucleic acid encoding a non-mammalian desaturase gene, said vector capable of transfecting cells of a non-human mammalian species. In a preferred embodiment, transfection of cells with said vector comprising said tissue-specific promoter results in transgenic animals which produce altered levels of long chain polyunsaturated fatty acids or essential fatty acids in the animals milk.

In one embodiment, the present invention contemplates a method, comprising: a) providing i) a non-human animal cell, ii) a vector comprising nucleic acid encoding a heterologous desaturase, and iii) a recipient non-human female animal; b) introducing said vector into said cell to create a transfected cell; c) transferring said transfected cell into said recipient female under conditions such that at least one offspring is produced, said offspring expressing said desaturase in one or more tissues.

In another embodiment, the present invention contemplates a method, comprising: a) providing i) a vector comprising a heterologous regulatory element in operable combination with a DNA sequence encoding a homologous desaturase, ii) a non-human animal cell and iii) a recipient non-human female animal; b) introducing said vector into said cell to create a transfected cell; c) transferring said transfected cell into said recipient female under conditions such that at least one offspring is produced, said offspring expressing said desaturase in one or more tissues. In a preferred embodiment, said heterologous regulatory element comprises a tissue-specific promoter which directs expression in mammary tissue and said offspring expresses said desaturase in said offspring's mammary tissue, resulting altered levels of long-chain polyunsaturated fatty acids in the offspring's milk.

In one embodiment, the present invention contemplates a method, comprising: a) providing i) a vector comprising a heterologous regulatory element in operable combination with a DNA sequence encoding a heterologous desaturase, ii) a non-human cell and iii) a recipient non-human female; b) introducing said vector into said cell to create a transfected cell; c) transferring said transfected cell into said recipient female under conditions such that at least one offspring is produced, said offspring expressing said desaturase in one or more tissues. In a preferred embodiment, said heterologous regulatory element comprises a tissue-specific promoter which directs expression in mammary tissue and said offspring expresses said desaturase in said offspring's mammary tissue, resulting in altered levels of long-chain polyunsaturated fatty acids in the offspring's milk.

In yet another embodiment, the present invention contemplates a method, comprising: a) providing i) a cell to be transfected selected from the group consisting of non-human embryonic stem (ES) cells, an fertilized egg or a cell of an early embryo, ii) a vector comprising a tissue-specific promoter in operable combination with a DNA sequence encoding a desaturase, iii) a recipient non-human female; b) introducing said vector into said cell to create a transfected cell; c) transferring said transfected cell into said recipient female under conditions such that at least one offspring is produced, said offspring expressing said desaturase in one or more tissues. In a preferred embodiment, said tissue-specific promoter directs expression in mammary tissue and said offspring expresses said desaturase in said offspring's mammary tissue, resulting in the secretion of long-chain polyunsaturated fatty acids in the offspring's milk.

It is not intended that the present invention be limited to particular desaturase genes. A variety of desaturase genes and sources of desaturase genes are contemplated. With regard to particular preferred desaturase genes, the present invention contemplates genes for Δ5 desaturase, Δ6 desaturase, Δ12 desaturase and Δ15 desaturase. With regard to sources of heterologous desaturase genes, the present invention contemplates a variety of sources, including but not limited to plant and fungal sources.

It is not intended that the present invention be limited to the uses for the essential fatty acids and LC-PUFAs generated by animal cells or transgenic animals which comprise the above-named heterologous desaturase genes. The present invention contemplates a variety of formulations comprising such essential fatty acids, their derivatives and downstream products, including but not limited to feed formulations, nutritional formulations and cosmetic formulations. Thus, for example, in one embodiment, the present invention contemplates a nutritional formulation comprising at least one essential fatty acid produced by one of the above-named transgenic animals or transfected cells.

It is not intended that, in making the above-named formulations, the present invention be limited to the recovery of essential fatty acids, their derivatives and downstream products from a particular bodily fluid or tissue. While milk is a convenient source, other bodily fluids containing essential fatty acids, their derivatives and downsteam products are contemplated including, but not limited to, urine. A preferred tissue source comprises animal fat.

The present invention also contemplates labelled essential fatty acids, derivatives and downstream products. Thus, for example, in one embodiment, the present invention contemplates an essential fatty acid produced by one of the above-named transgenic animals or transfected cells, said essential fatty acid comprising a reporter molecule. Suitable reporter molecules or labels include radiolabels, enzymes, fluorescent, chemiluminescent, or chromogenic agents. Such labelled fatty acids, derivatives and/or downstream products can be used diagnostically by introducing them to cells in culture (including but not limited to tumor cells).

It is not intended that the present invention be limited to particular non-human animals. Animals of all types are contemplated, including but not limited to insects, nematodes, fish, birds and mammals. A variety of preferred animals are contemplated, including but not limited to mice, rats, rabbits, pigs, goats, sheep, cows and horses.

### BRIEF DESCRIPTION OF THE FIGURES

FIG. 1 is a schematic showing Fatty Acid metabolism in Mammalian Tissues.

FIG. 2A-D schematically shows the construction of the eukaryotic desaturase expression vectors.

FIG. 2A schematically shows the construction of the pCMV-Δ6-bGH plasmid.

FIG. 2B schematically shows the construction of the pCMV-Δ12-bGH plasmid.

FIG. 2C schematically shows the construction of the pWAP-Δ6-bGH plasmid.

FIG. 2D schematically shows the construction of the pWAP-Δ12-bGH plasmid.

FIG. 3 is a bar graph depicting the % Linoleic acid (18:2n-6) levels in control and Δ12-desaturase gene-transfected L cells.

FIG. 4 is a bar graph depicting the % Eicosadienic acid levels (20:2n-6) levels in control and Δ12-desaturase gene-transfected L cells.

FIG. 5 is a bar graph depicting the Fatty acid profiles in control and Δ6-desaturase gene-transfected L cells.

FIG. 6 depicts the nucleotide sequence of fungal Δ5 desaturase (SEQ ID: 1).

FIG. 7 depicts the amino acid sequence of fungal Δ5 desaturase (SEQ ID: 2).

FIG. 8 depicts the nucleotide sequence of fungal Δ6 desaturase (SEQ ID: 3).

FIG. 9 depicts the amino acid sequence of fungal Δ6 desaturase (SEQ ID: 4).

FIG. 10 depicts the nucleotide sequence of fungal Δ12 desaturase (SEQ ID: 5).

FIG. 11 depicts the amino acid sequence of fungal Δ12 desaturase (SEQ ID: 6).

FIG. 12 schematically depicts downstream products of arachidonic acid in the 5-lipoxygenase pathway.

FIG. 13 schematically depicts downstream products of arachidonic acid in the 12-lipoxygenase pathway.

FIG. 14 schematically depicts downstream products of arachidonic acid in the 15-lipoxygenase pathway.

FIG. 15 schematically depicts downstream products of arachidonic acid in the cytochrome P-450 pathway.

FIG. 16 schematically depicts downstream products of arachidonic acid in the cyclooxygenase pathway (pathway I).

FIG. 17 schematically depicts downstream products of arachidonic acid in the cyclooxygenase pathway (pathway II).

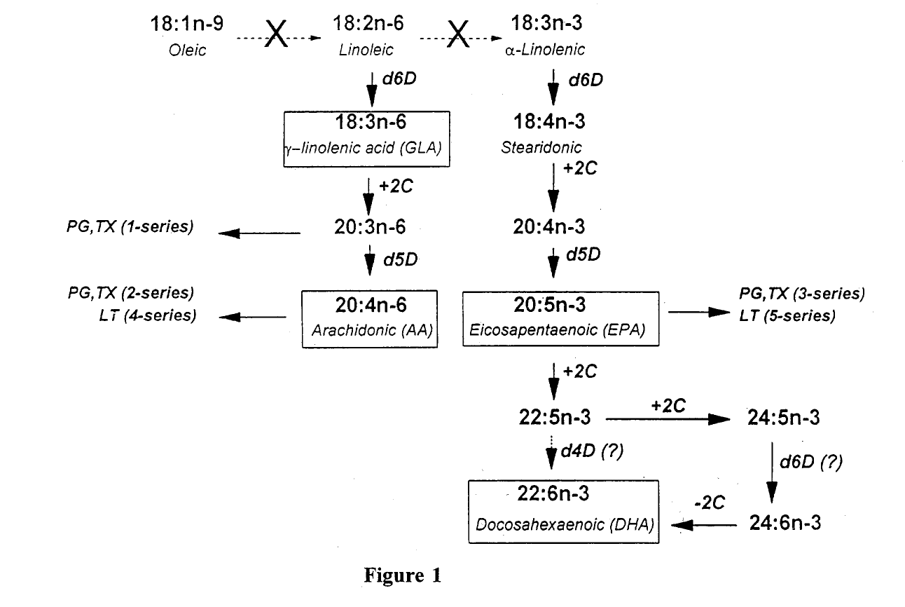

### DEFINITIONS

To facilitate understanding of the invention, a number of terms are defined below.

The abbreviations used herein are: LC-PUFAs, Long chain-poly-unsaturated fatty acids; PG, prostaglandins; LT, Leukotrienes; GLA, γ-linolenic acid; DGLA, dihomo-γ-linolenic acid; AA, Arachidonic Acid; EPA, Eicosapentaenoic acid; DHA, Docosahexaenoic acid; SDA, stearidonic acid; FA, Fatty acids, EFA, essential fatty acids; d5D, Δ5 desaturase; d6D, Δ6 desaturase; d12D, Δ12 desaturase; TX, Thromboxanes.

The term “LC-PUFA” as used herein refer to fatty acids with chain lengths beyond 18-carbons that has two or more double bonds. A large number of such LC-PUFA (but not all) can be derived from Linoleic Acid (18:2n-6) and a-Linolenic Acid (18:3n-3), the two so-called “essential fatty acids.”

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “portion” when used in reference to a nucleotide sequence refers to fragments of that nucleotide sequence. The fragments may range in size from 5 nucleotide residues to the entire nucleotide sequence minus one nucleic acid residue.

The term “recombinant DNA molecule” as used herein refers to a DNA molecule which is comprised of segments of DNA joined together by means of molecular biological (i.e., non-naturally occurring) techniques.

As used herein, the terms “vector” and “vehicle” are used interchangeably in reference to nucleic acid molecules that transfer DNA segment(s) from one cell to another.

The term “expression vector” or “expression cassette” as used herein refers to a recombinant DNA molecule containing a desired coding sequence and appropriate nucleic acid sequences necessary for the expression of the operably linked coding sequence in a host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “in operable combination”, “in operable order” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

As used herein, the terms “complementary” or “complementarity” are used in reference to “polynucleotides” and “oligonucleotides” (which are interchangeable terms that refer to a sequence of nucleotides) related by the base-pairing rules. For example, for the sequence 5′-AGT-3′ is complementary to the sequence 5′-ACT-3′. Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands.

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

Low stringency conditions comprise conditions equivalent to binding or hybridization at 42° C. in a solution consisting of 5×SSPE (43.8 g/l NaCl, 6.9 g/l NaH₂PO₄•H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharmacia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target ( DNA, RNA, base composition, present in solution or immobilized, etc.) and the concentration of the salts and other components (e.g., the presence or absence of formamide, dextran sulfate, polyethylene glycol), as well as components of the hybridization solution may be varied to generate conditions of low stringency hybridization different from, but equivalent to, the above listed conditions. In addition, the art knows conditions which promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe which can hybridize to either or both strands of the double-stranded nucleic acid sequence under conditions of low stringency as described above.

When used in reference to a single-stranded nucleic acid sequence, the term “substantially homologous” refers to any probe which can hybridize (i.e., it is the complement of) to the single-stranded nucleic acid sequence under conditions of low stringency as described above.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (as determined, e.g., by C₀t or R₀t analysis) or between one nucleic acid sequence present in solution and another nucleic acid sequence immobilized to a solid support [e.g., a nylon membrane or a nitrocellulose filter as employed in Southern and Northern blotting, dot blotting or a glass slide as employed in in situ hybridization, including FISH (fluorescent in situ hybridization)].

“Stringency” when used in reference to nucleic acid hybridization typically occurs in a range from about Tₘ−5° C. (5° C. below the Tₘ of the probe) to about 20° C. to 25° C. below Tₘ. The term “Tₘ” is used in reference to the “melting temperature.” The melting temperature is the temperature at which a population of double-stranded nucleic acid molecules becomes half dissociated into single strands. As will be understood by those of skill in the art, a stringent hybridization can be used to identify or detect identical polynucleotide sequences or to identify or detect similar or related polynucleotide sequences. Under “stringent conditions” a sequence or fragments thereof will hybridize to the sequence's exact complement and closely related sequences.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “an oligonucleotide having a nucleotide sequence encoding a gene” means a nucleic acid sequence comprising the coding region of a gene, i.e., the nucleic acid sequence which encodes a gene product. The coding region may be present in either a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc.

Transcriptional control signals in eukaryotes comprise “promoter” and “enhancer” elements. Promoters and enhancers consist of short arrays of DNA sequences that interact specifically with cellular proteins involved in transcription [Maniatis, T. et al., Science 236:1237 (1987)]. Promoter and enhancer elements have been isolated from a variety of eukaryotic sources including genes in plant, yeast, insect and mammalian cells and viruses (analogous control elements, i.e., promoters, are also found in prokaryotes). The selection of a particular promoter and enhancer depends on what cell type is to be used to express the protein of interest. The term “promoter sequence” as used herein refers to a single promoter sequence as well as to a plurality (i.e., one or more) of promoter sequences which are operably linked to each other and to at least one DNA sequence of interest. For example, one of skill in the art knows that it may be desirable to use a double promoter sequence (i.e., a DNA sequence containing two promoter sequences) or a triple promoter sequence (i.e., a DNA sequence containing three promoter sequences) to control expression of a DNA sequence of interest.

The presence of “splicing signals” on an expression vector often results in higher levels of expression of the recombinant transcript. Splicing signals mediate the removal of introns from the primary RNA transcript and consist of a splice donor and acceptor site [Sambrook, J. et al., Molecular Cloning: A Laboratory Manual, 2nd ed., Cold Spring Harbor Laboratory Press, New York (1989) pp. 16.7-16.8]. A commonly used splice donor and acceptor site is the splice junction from the 16S RNA of SV40. Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which is isolated from one gene and placed 3′ of another gene.

The terms “cognate promoter” and “cognate promoter of RNA polymerase” refer to a promoter sequence which is a naturally occurring promoter sequence in a gene encoding the RNA polymerase. For example, the cognate promoter of T₇ RNA polymerase is the promoter which is derived from the gene encoding RNA polymerase in T₇ bacteriophage. The cognate promoter sequence may be cloned from the genome encoding the RNA polymerase. The location of a promoter may be identified by approaches and methods well known in the art, including DNase footprinting of the RNA polymerase-bound genome DNA, mutational analysis, etc. Alternatively, where the sequence of a cognate promoter is known, the promoter sequence may be synthesized.

The term “transfection” as used herein refers to the introduction of a transgene into a cell. The term “transgene” as used herein refers to any nucleic acid sequence which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence which is naturally found in the cell into which it is introduced so long as it does not contain some modification (e.g., a point mutation, the presence of a selectable marker gene, etc.) relative to the naturally-occurring sequence. The term “heterologous DNA sequence” refers to a nucleotide sequence which is not endogenous to the cell into which it is introduced. Heterologous DNA includes a nucleotide sequence which is ligated to, or is manipulated to become ligated to, a nucleic acid sequence to which it is not ligated in nature, or to which it is ligated at a different location in nature. Heterologous DNA also includes a nucleotide sequence which is naturally found in the cell into which it is introduced and which contains some modification relative to the naturally-occurring sequence. Heterologous DNA encodes RNA and proteins that are not normally produced by the cell into which it is introduced. An example of a heterologous DNA of the present invention comprises a nucleotide sequence which encodes a desaturase which is not found in the mammalian cell into which it is introduced. Another example of the present invention is a desaturase gene which is ligated to a promoter sequence to which it is not naturally ligated.

Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, biolistics (i.e., particle bombardment) and the like.

The term “stable transfection” or “stably transfected” refers to the introduction and integration of a transgene into the genome of the transfected cell. The term “stable transfectant” refers to a cell which has stably integrated one or more transgenes into the genomic DNA.

The term “transient transfection” or “transiently transfected” refers to the introduction of one or more transgenes into a transfected cell in the absence of integration of the transgene into the host cell's genome. The term “transient transfectant” refers to a cell which has transiently integrated one or more transgenes.

A “transgenic organism” as used herein refers to an organism in which one or more cells has been transiently transfected or stably transfected with a transgene by experimental manipulation. Transgenic organisms may be produced by several methods including the introduction of a “transgene” comprising nucleic acid (usually DNA) into an embryonic target cell or a somatic target cell (such as cells of the mammary gland) of a non-human organism by way of human intervention.

“Insertion” is used to refer to the process whereby a portion of heterologous DNA or a heterologous gene that is introduced into the genome of a host. The DNA which is inserted is referred to as an “insert”.

The terms “transgenic mammal” or “transgenic host” are used to refer to a mammal or cell which has had a transgene inserted into its genome. As a result of this insertion, the transgenic host produces heterologous biological material that it would not normally synthesize. Heterologous entities are present or are produced by a transgenic host as a result of the insertion of foreign genetic material into the host cell genome.

The term “primary gene product” refers to a biological entity which is formed directly as a result of the transcription and translation of a homologous or heterologous gene. Examples thereof include proteins, antibodies, enzymes and the like.

The term “secondary gene product” refers to a product which is formed as a result of the biological activity of a primary gene product. An example thereof, is PUFAs which are formed as a result of the expression of specific desaturases.

The term “products” or “biological products” refer to products produced or synthesized by a transgenic animal as a result of the insertion of a transgene into the genome of the animal. More specifically, the term means biological products which are secondary gene products or other downstream products altered by transgene expression. One example hereof, as described below, is LC-PUFAs produced by transgenic mice.

### GENERAL DESCRIPTION OF THE INVENTION

The invention generally relates to compositions and methods of synthesis of essential fatty acids and their derivatives, long chain poly-unsaturated fatty acids and eicosanoids in transfected cells and in transgenic animals. The ability to produce long chain fatty acids derived from Linoleic Acid (18:2n-6) and α-Linolenic Acid (18:3n-3) in cultured cells and in transgenic animals has far reaching economic and scientific implications. Arachidonic Acid and γ-Linolenic Acid (GLA) are important biologically active molecules in and of themselves. Additionally, they serve as precursors for the synthesis of Eicosanoids, such as Prostaglandins, Thromboxanes, Prostacyclins, and Leukotrienes, molecules that again have been shown to possess various biological activities. Also, the ability to produce Eicosapentaenoic Acid (EPA) and Docosahexaeonic Acid (DHA) in mammalian cells and transgenic animals have important implications since these molecules have been show to be potent biologically active molecules.

The Description of the invention involves: A) Selecting Transgenes: Desaturases and the Construction of Expression Vectors Comprising the Transgenes; B) Introduction of the Expression Construct into a Particular Cell; C) Transgenic Animals and Methods of Introduction of Transgenes; D) Tissue-Specific Expression of Transgenes and Detection of the Expression Construct; and E) Fatty acid Production, including Prostaglandins, Prostacyclins, Thromboxanes and Leukotrienes, in Cell Lines and Bioreactors.

A. Selecting Transgenes: Desaturases and the Construction of Expression Vectors Comprising the Transgenes

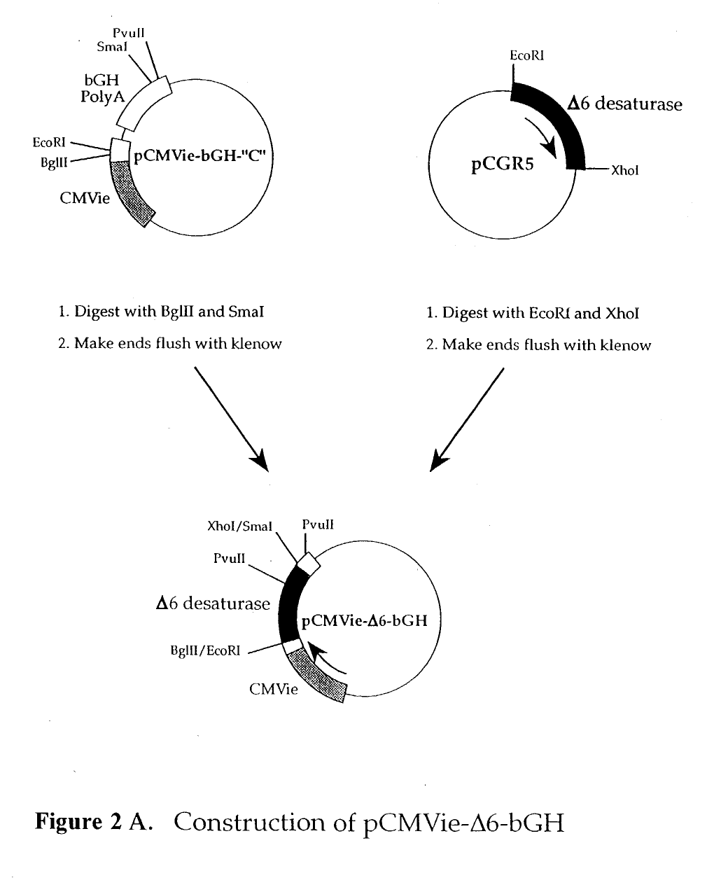

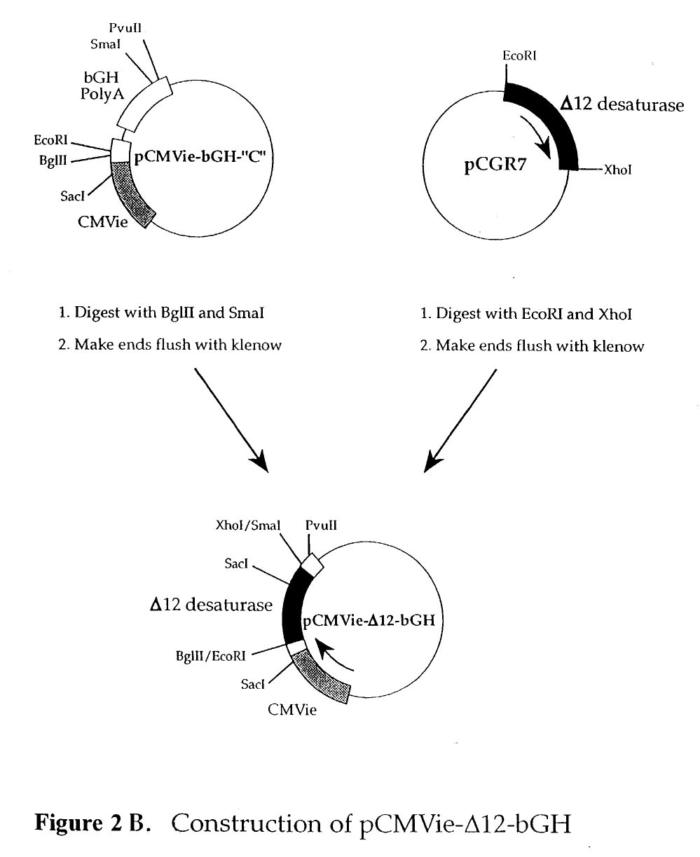

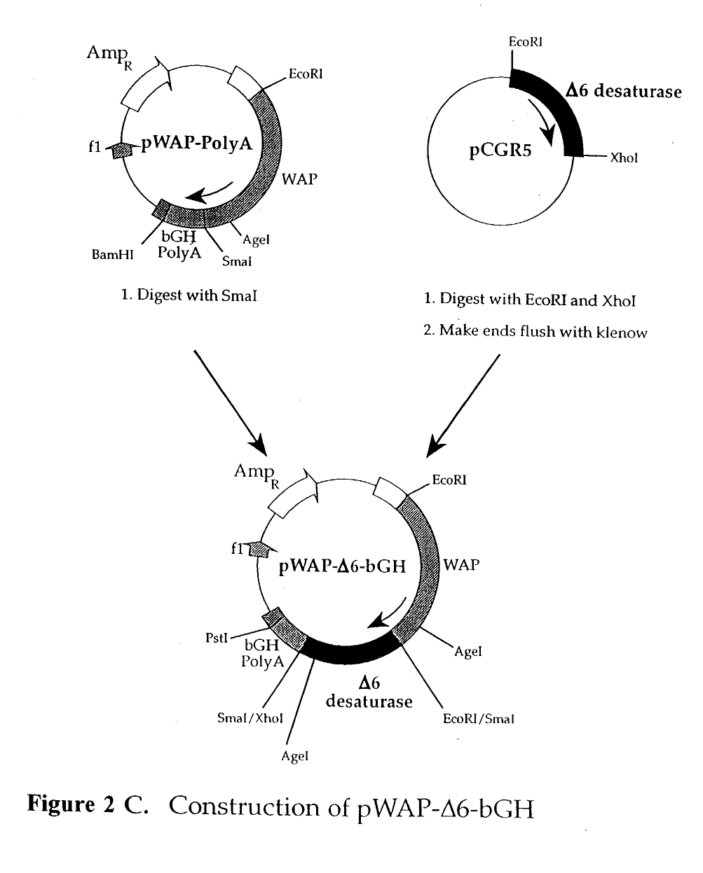

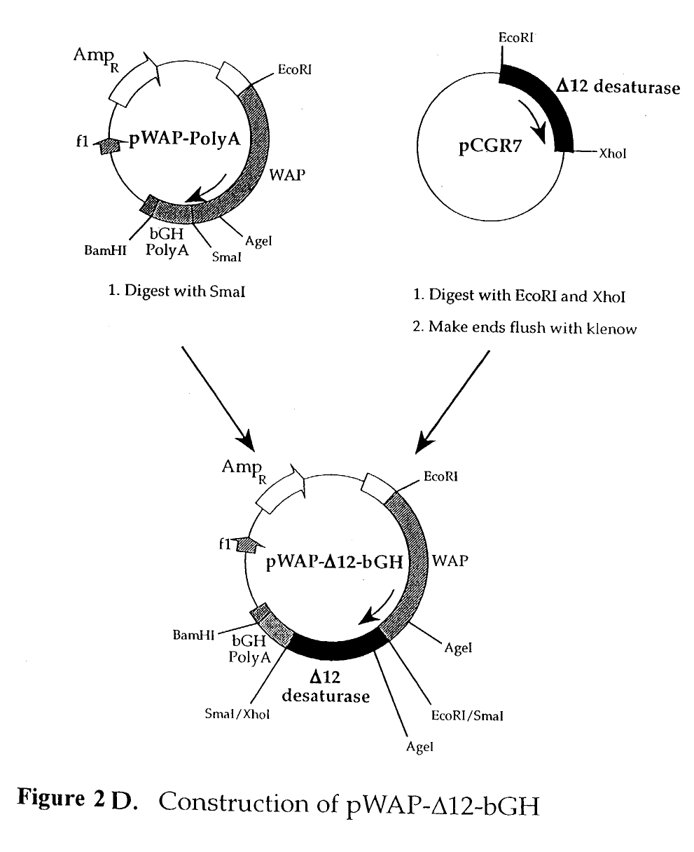

The present invention contemplates introducing desaturase genes into mammalian cells. It is not intended that the present invention be limited to one source or one type of desaturase gene. In one embodiment, a fungal desaturase gene is contemplated. For this embodiment, a 1,382 bp EcoRI-XhoI DNA fragment encoding the Δ6-desaturase gene (SEQ ID: 3) was isolated from plasmid pCGR5 and ligated into plasmid pCMV-BGH-C, [A. Martin-Gallardo et al., “A comparison of bGH expression in mouse L cells directed by the Moloney murine leukemia virus long terminal repeat, the simian virus 40 early or cytomegalovirus immediate early promotors,” Gene 70:151-156 (1988)], which had been cleaved with BglII and SmaI. The termini of the DNA molecules were made flush using Klenow polymerase prior to ligation. The resulting plasmid, pCMVie-Δ6-bGH, utilizes the cytomegalovirus immediate early transcriptional regulatory element to direct Δ-6-desaturase transcription and the bGH polyadenylation signal for proper processing of the 3′ terminus of desaturase mRNA (See FIG. 2A). Similarly, a 1,209 bp EcoRI-XhoI DNA fragment encoding the Δ12-desaturase gene(SEQ ID: 5) was isolated from plasmid pCGR7 and ligated into plasmid pCMV-BGH-C which had been cleaved with BglII and SmaI to generate the plasmid, pCMVie-Δ12-bGH (See FIG. 2B). The termini of these DNA molecules were also made flush using Klenow polymerase prior to ligation.

The DNA fragments encoding the Δ6-desaturase gene and the Δ12-desaturase gene were also ligated into plasmid, pWAP-polyA which had been cleaved with SmaI (See FIGS. 2C and 2D). The resulting plasmids, pWap-Δ6-bGH and pWap-Δ12-bGH utilize the murine whey acidic protein transcriptional regulatory element to direct Δ6 and Δ12-desaturase transcription and the bGH polyadenylation signal for proper processing of the 3′ terminus of desaturase mRNAs.

B. Introduction of the Expression Construct Into a Particular Cell

In order to bring about tissue specific and/or cell type specific expression in transgenic animals, the expression vector which contains the murine whey acidic protein transcriptional regulatory element is in operable combination with the nucleic acid sequences encoding Δ5-desaturase, Δ6-desaturase, Δ12-desaturase or Δ15-desaturase sequences of the invention. Where the desaturase is to be transfected into a host cell (such as a cell in culture), the CMV promoter can be used. Host cells include bacterial, yeast, plant, insect, and mammalian cells. In a preferred embodiment the host cell is mammalian. In a more preferred embodiment, the host cell is a mouse cell.

Any number of selection systems may be used to recover transfected cell lines. These include, but are not limited to, the herpes simplex virus thymidine kinase (Wigler M et al. (1977) Cell 11:223-32) and adenine phosphoribosyltransferase (Lowy I et al. (1980) Cell 22:817-23) genes which can be employed in tk⁻ or aprt⁻ cells, respectively. Also, antimetabolite, antibiotic or herbicide resistance can be used as the basis for selection; for example, dhfr which confers resistance to methotrexate [Wigler M et al., (1980) Proc Natl Acad Sci 77:3567-70]; npt, which confers resistance to the aminoglycosides neomycin and G-418 [Colbere-Garapin F et al., (1981) J. Mol. Biol. 150:1-14] and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine [Hartman SC and RC Mulligan (1988) Proc Natl Acad Sci 85:8047-51]. Recently, the use of a reporter gene system which expresses visible markers has gained popularity with such markers as β-glucuronidase and its substrate (GUS), luciferase and its substrate (luciferin), and β-galactosidase and its substrate (X-Gal) being widely used not only to identify transformants, but also to quantify the amount of transient or stable protein expression attributable to a specific vector system [Rhodes CA et al. (1995) Methods Mol Biol 55:121-131].

Although the presence or expression of the reporter gene usually indicates the presence or expression, respectively, of the tandem heterologous nucleic acid sequence as well. However, it is preferred that the presence and expression of the desired heterologous nucleic acid sequence be confirmed. This is accomplished by procedures known in the art which include DNA-DNA or DNA-RNA hybridization or amplification using probes, or fragments of the heterologous nucleic acid sequence. For example, Fluorescent In Situ Hybridization (FISH) can be used to detect the heterologous nucleic acid sequence in cells. Several guides to FISH techniques are available, e.g., Gall et al. Meth. Enzymol. 21:470-480 (1981); Angerer et al., in “Genetic Engineering: Principles and Methods,” Setlow & Hollaender, Eds. Vol. 7 pp. 43-65, Plenum Press, New York (1985). Alternatively, DNA or RNA can be isolated from cells for detection of the transgene by Southern or Northern hybridization or by amplification based assays. Nucleic acid amplification based assays involve the use of oligonucleotides or oligomers based on sequence of the nucleic acid sequence of interest in order to detect cells and tissues which contain the DNA or RNA encoding the transgene of interest. As used herein, the terms “oligonucleotides” and “oligomers” refer to a nucleic acid sequence of at least about 10 nucleotides and as many as about 60 nucleotides, preferably about 15 to 30 nucleotides, and more preferably about 20-25 nucleotides, which can be used as a probe or amplimer. Standard PCR methods useful in the present invention are described by Innis et al. (Eds.), “PCR Protocols: A Guide to Methods and Applications,” Academic Press, San Diego (1990)].

Yet another alternative for the detection of heterologous nucleic acid sequences is by detecting the polypeptide product of transcription of the heterologous nucleotide sequence. A variety of protocols which employ polyclonal or monoclonal antibodies specific for the protein product are known in the art. Examples include enzyme-linked immunosorbent assay (ELISA), radioimmunoassay (RIA) and fluorescent activated cell sorting (FACS). A competitive binding assay may also be used. Alternatively, a two-site, monoclonal-based immunoassay which utilizes monoclonal antibodies that are reactive to two non-interfering epitopes on the protein of interest may be employed. These and other assays are described in, among other places, R. Hampton et al., Serological Methods a Laboratory Manual, APS Press, St Paul Minn. (1990) and D. E. Maddox et al., J. Exp. Med. 158:1211(1983).

A wide variety of labels and conjugation techniques are known by those skilled in the art and can be used in various nucleic acid and amino acid assays. Means for producing labeled hybridization or PCR probes for detecting related sequences include oligolabeling, nick translation, end-labeling or PCR amplification using a labeled nucleotide. Alternatively, the nucleic acid sequence of interest, or any portion of it, may be cloned into a vector for the production of an mRNA probe. Such vectors are known in the art, are commercially available, and may be used to synthesize RNA probes in vitro by addition of an appropriate RNA polymerase such as T7, T3 or SP6 and labeled nucleotides. A number of companies such as Pharmacia Biotech (Piscataway N.J.), Promega (Madison Wis.), and US Biochemical Corp (Cleveland Ohio.) supply commercial kits and protocols for these procedures. Suitable reporter molecules or labels include those radionuclides, enzymes, fluorescent, chemiluminescent, or chromogenic agents as well as substrates, cofactors, inhibitors, magnetic particles and the like.

In specific embodiments of the present invention, stably transfected L cells were generated using the calcium phosphate precipitation method as previously described [M. Wigler et al., “Transfer of purified herpes virus thymidine kinase gene to cultured mouse cells,” Cell 11:223-232 (1977); A. Pellicer et al., “Altering genotype and phenotype by DNA-mediated gene transfer,” Science 209:1414-1422 (1980); B. Wold et al., “Introduction and expression of a rabbit β-globin gene in mouse fibroblasts,” Proc. Nat. Acad. Sci. USA 76:5684-5688 (1979); J. M. Roberts and R. Axel, “Gene amplification and gene correction in somatic cells,” Cell 29:109-119 (1982)]. This method utilizes a plasmid DNA (pD1AT3) which encodes two selectable markers: an intact APRT gene and a truncated TK gene lacking transcriptional regulatory (enhancer) sequences.

In other specific embodiments, stably transfected Hela cells were generated using the Lipofectamine Reagent method (BRL, Gaithersburg, Md.) and cells were selected that exhibited neomycin resistance. The neomycin resistant Hela cell pool was then expanded and analyzed further.

C. Transgenic Animals and Methods of Introduction of Transgenes

A first step in the generation of the transgenic animals, is the introduction of a construct containing the desired heterologous nucleic acid sequence such as the Δ12-desaturase, Δ6-desaturase, or the Δ5-desaturase into target cells. Several methods are available for introducing the expression vector which contains the heterologous nucleic acid sequence into a target cell, including microinjection, retroviral infection, and implantation of embryonic stem cells. These methods are discussed as follows.

i. Microinjection Methods

Direct microinjection of expression vectors into pronuclei of fertilized eggs is the preferred, and most prevalent, technique for introducing heterologous nucleic acid sequences into the germ line [Palmiter (1986) Ann. Rev. Genet. 20:465-499]. Technical aspects of the microinjection procedure and important parameters for optimizing integration of nucleic acid sequences are known to the art [Brinster et al., (1985) Proc. Natl. Acad. Sci. USA 82:4438-4442; Gordon et al., (1983) Meth. Enzymol. 101:411-433; Hogan et al., (1986) Manipulation of the Mouse Embryo: A Laboratory Manual. Cold Spring Harbor, New York: Cold Spring Harbor Lab.].

Once the expression vector has been injected into the fertilized egg cell, the cell is implanted into the uterus of a pseudopregnant female and allowed to develop into an animal. Of the founder transgenic animals born, 70% carry the expression vector sequence in all of their cells, including the germ cells. The remaining 30% of the transgenic animals are chimeric in somatic and germ cells because integration of the expression vector sequence occurs after one or more rounds of replication. Heterozygous and homozygous animals can then be produced by interbreeding founder transgenics. This method has been successful in producing transgenic mice, sheep, pigs, rabbits and cattle [Jaenisch (1988) supra; Hammer et al., (1986) J. Animal Sci.: 63:269; Hammer et al., (1985) Nature 315:680-683; Wagner et al., (1984) Theriogenology 21:29].

ii. Retroviral Methods

Retroviral infection of preimplantation embryos with genetically engineered retroviruses may also be used to introduce transgenes into an animal cell. For example, blastomeres have been used as targets for retroviral infection [Jaenisch, (1976) Proc. Natl. Acad. Sci USA 73:1260-1264]. Transfection is typically achieved using a replication-defective retrovirus carrying the transgene [Jahner et al., (1985) Proc. Natl. Acad. Sci. USA 82:6927-6931; Van der Putten et al., (1985) Proc. Natl. Acad Sci USA 82:6148-6152]. Transfection is obtained, for example, by culturing eight-cell embryos, from which the zona pellucida has been removed with fibroblasts which produce the virus [Van der Putten (1985), supra; Stewart et al., (1987) EMBO J. 6:383-388]. The transfected embryos are then transferred to foster mothers for continued development. Alternatively, infection can be performed at a later stage. Virus or virus-producing cells can be injected into the blastocoele [Jahner et al., (1982) Nature 298:623-628]. Yet another alternative method involves intrauterine retroviral infection of the midgestation embryos [Jahner et al. (1982), supra].

The advantages of retroviral infection methods include the ease of transfection and the insertion of a single copy of the transgene, which is flanked by the retroviral long terminal repeats (LTRs), into the chromosome. However, this method is not a preferred method because most of the founders will show mosaicism since infection occurs after cell division has begun. This necessitates outbreeding to establish homozygous and heterozygous lines suitable for analysis of gene expression.

iii. Embryonic Stem Cell Implantation

Another method of introducing transgenes into the germ line involves using embryonic stem (ES) cells as recipients of the expression vector. ES cells are pluripotent cells directly derived from the inner cell mass of blastocysts [Evans et al., (1981) Nature 292:154-156; Martin (1981) Proc. Natl. Acad Sci. USA 78:7634-7638; Magnuson et al., (1982) J. Embryo. Exp. Morph. 81:211-217; Doetchman et al., (1988) Dev. Biol. 127:224-227], from inner cell masses [Tokunaga et al., (1989) Jpn. J. Anim. Reprod. 35:113-178], from disaggregated morulae [Eistetter, (1989) Dev. Gro. Differ. 31:275-282] or from primordial germ cells [Matsui et al., (1992) Cell 70:841-847; Resnick et al., (1992) Nature 359:550-551]. Expression vectors can be introduced into ES cells using any method which is suitable for gene transfer into cells, e.g., by transfection, cell fusion, electroporation, microinjection, DNA viruses, and RNA viruses [Johnson et al., (1989) Fetal Ther. 4 (Suppl. 1): 28-39].

The advantages of using ES cells include their ability to form permanent cell lines in vitro, thus providing an unlimited source of genetic material. Additionally ES cells are the most pluripotent cultured animal cells known. For example, when ES cells are injected into an intact blastocyst cavity or under the zona pellucida, at the morula stage embryo, ES cells are capable of contributing to all somatic tissues including the germ line in the resulting chimeras.

Once the expression vector has been introduced into an ES cell, the modified ES cell is then introduced back into the embryonic environment for expression and subsequent transmission to progeny animals. The most commonly used method is the injection of several ES cells into the blastocoel cavity of intact blastocysts [Bradley et al., (1984) Nature 309:225-256]. Alternatively, a clump of ES cells may be sandwiched between two eight-cell embryos [Bradley et al., (1987) in “Teratocarcinomas and Embryonic Stem Cells: A Practical Approach,” Ed. Robertson E. J. (IRL, Oxford, U.K.), pp. 113-151; Nagy et al., (1990) Development 110:815-821]. Both methods result in germ line transmission at high frequency.

Target cells which contain the heterologous nucleic acid sequences are recovered, and the presence of the heterologous nucleic acid sequence in the target cells as well as in the animal is accomplished as described supra.

D. Tissue-Specific Expression and Detection of Transgenes

The present invention provides methods for selectively expressing a nucleotide sequence of interest in a particular cell type and/or a particular tissue. The transfected animal cell is allowed to develop into a transgenic animal in which the nucleotide sequence of interest, i.e, the Δ6 and/or Δ12-desaturase genes is expressed selectively in a particular tissue such as the mammary glands. The expression vectors comprising the desaturase sequences of the present invention, pWap-Δ6-bGH and pWap-D12-bGH utilize the murine whey acidic protein transcriptional regulatory element to direct Δ6 and Δ12-desaturase transcription and the bGH polyadenylation signal for proper processing of the 3′ terminus of desaturase mRNAs. In addition, these are under the control of the murine whey acidic protein transcriptional regulatory element, that directs gene expression primarily to the lactating mammary gland tissue.

The selective expression of the gene of interest (i.e., the desaturase transgenes) in tissues and cells of transgenic animals may be determined using several methods known in the art. For example, expression of mRNA encoded by the gene of interest may be determined by using in situ hybridization. This involves synthesis of an RNA probe which is specific for a portion of or the entire gene of interest, e.g., by using PCR. The PCR amplified fragment is subcloned into a plasmid (e.g., pBluescript (Stratagene)) and the RNA probe synthesized using labelled UTP (e.g., ³⁵S-UTP) and RNA polymerase (e.g., T3 or T7 polymerase (Promega)). Paraffin-embedded tissue sections are mounted on slides, deparaffinized, rehydrated and the protein digested (e.g., with proteinase K), then dehydrated prior to hybridization with the RNA probe at the desired hybridization stringency. Slides are then developed for autoradiography using commercially available developers. Labelling of tissues and cells as detected on the autoradiographs indicates expression in those tissues and cells of the mRNA encoded by the gene of interest.

Alternatively, expression of the protein product of the gene of interest may be determined using immunohistochemical techniques. Briefly, paraffin-embedded tissue sections are dewaxed, rehydrated, treated with a first antibody which is specific for the polypeptide product of the gene of interest. Binding is visualized, for example, by using a secondary biotinylated antibody which is specific for the constant region of the primary antibody, together with immunoperoxidase and 3,3′-amiobenzidine as a substrate. Sections may then be stained with hematoxylin to visualize the cellular histology. Antibody binding of tissues and cells which is detected by antibody binding demonstrates expression of the protein product of the gene of interest in these tissues and cells.

E. Fatty Acid Production, Including Prostaglandins and Leukotrienes in Cell Lines and Bioreactors

i. LC-PUFA Production

The present invention provides alternate methods for altering or increasing the production of LC-PUFAs and derivative products by use of mammalian cells and transgenic animals into which has been inserted genes or cDNAs encoding desaturases.

In particular, the present invention provides a method to generate mammalian cells in which fungal delta-12, delta-6 and delta-5 genes are expressed. The transfected cells exhibited enhanced production of both n-6 and n-3 LC-PUFAs, as they had obtained the ability to synthesize essential fatty acids or utilized the exogenous essentail fatty acids (EFA) as precursor for production of LC-PUFA. In addition, the desaturase genes were introduced into animals, gene expression targeted specifically to the mammary glands, generating transgenic mice expressing delta-12, delta-6 or delta-5 genes. The milk derived from the transgenic mice contained a significantly higher level of LC-PUFA than that from the control mice.

Thus, the present invention provides alternative sources of EFA and LC-PUFA in commonly used commodities, such as milk, infant formula, dietary supplement and pharmaceuticals. In addition, the methods of the present invention provide an alternate source for the generation of eicosanoids. Eicosanoids (prostaglandins, leukotrienes, and lipoxins), oxygenated lipids, and Platelet Activating Factor remain the focus of rational drug design targets given their established roles in cell-cell communication and as mediators in inflammation and pathophysiologic events. Identification of key enzymes in these pathways are implicating involvement of the nuclear membrane at the functional level. Results from transgenic animals have identified novel bioactive eicosanoids, including 15-epi-lipoxins, isoprostanes, and isoleukotrienes, that offer new concepts to consider in formation of Lipid-derived mediators (LM) and the actions of nonsteroidal anti-inflammatory drugs. These findings indicate that LM play critical and essential roles in both signal transduction and cell-cell communication and will continue to be important pathways to be considered in novel therapeutic approaches (See Serhan, C. N. et al., Lipid mediator networks in cell signaling: update and impact of cytokines, FASEB J. 10: 1147-1158, 1996).

ii. Cell Lines as a Source of Essential FA's or Altered Levels of LC-PUFAs

The present invention provides Mammalian cells (including but not limited to Human HeLa cells, Mouse L cells) transfected with fungal Δ12 and/or Δ6, Δ5 desaturase genes, that exhibits enhanced production of both w6 and w3 LC-PUFAs. While an understanding of a precise mechanism is not necessary to the successful use of the invention, it is believed that this is because the transfected cells have obtained the ability to synthesize essential fatty acids (EFA), or utilize the exogenous EFA as precursors for production of LC-PUFAs. The minimum number of genes required for increased LC-PUFA production varied between different cell lines, e.g. human Hela cells required only Δ12 desaturase.

ii. Cells in Bioreactors

In some embodiments, the present invention contemplates the use of mammalian cells in bioreactors, for the large scale production of LC-PUFAs [see U.S. Pat. No. 5,459,069 to Palsson et al. and U.S. Pat. No. 5,563,068 to Zhang et al., both hereby incorporated by reference]. Some bioreactors utilize hollow fiber systems. Frequently, bundles of parallel fibers are enclosed in an outer compartment; cells are grown on the outside surface of the fibers, while nutrient- and gas-enriched medium flows through the center of the hollow fibers, nourishing the cells [see, e.g., U.S. Pat. No. 5,512,474 to Clapper et al., hereby incorporated by reference].

In addition, bioreactors utilizing microcarriers (e.g., DEAE-derivatived dextran beads) can be used in conjunction with the present invention. In preferred embodiments, cell adhesion proteins like collagen, fibronectin, and laminin are used to anchor the cells to the solid support; collagen is the most preferred cell adhesion protein. Microcarriers may also incorporate an ionic charge to assist in cell attachment to the microcarrier. Frequently, the microcarriers are porous beads that are sufficiently large to allow cells to migrate and grow in the interior of the bead [see U.S. Pat. No. 5,512,474 to Clapper et al.].

iv. Transgenic Animals as a Source of EFAs And for Enhanced Levels of PUFAs

The present invention provides the first, novel transgenic animals that express specific desaturase transgenes. By expressing a Δ12-desaturase (enzyme), products of the omega 6 pathway (that is GLA and Arachdonic Acid), were hoped for. However, surprisingly, the omega three series of LC-PUFAs, in particular EPA, DPA, and DHA were obtained in the transgenic animals. According to the information available in the current literature, these products are unexpected from Δ12-desaturase gene expression.

As mentioned in U.S. Pat. No. 5,689,050, hereby incorporated by reference, for expression data concerning transgenic organisms which express desaturases, the Δ12- and Δ6-desaturases must be co-expressed in order to obtain the desired products. For example, expression of Δ12-desaturase by plants yields Linoleic Acid from Oleic Acid. Plants cannot convert linoleic acid to other LC-PUFAs, since it lacks the enzymatic machinery to do so. Also, if one wanted to produce GLA in plants, the Δ6-desaturase enzyme needs to be inserted (to convert Linoleic acid to GLA) along with Δ12-desaturase. Thus, in plants, one needs to insert each and every enzyme of each metabolic conversion in order to obtain the desired results. However, based on the surprising results described herein, the latter may not be true for animals. Animal cells can convert linoleic acid to various LC-PUFA intermediates and products. But the exact pathways are not specifically and completely known. One would not have anticipated obtaining the omega 3 series of products from expression of Δ12-desaturase in animal cells, and also predicted that Δ12-, and Δ5-desaturase would need to be inserted in order to obtain EPA, DPA, and DHA. However, this was not the case. Thus, it is exemplary that essentially what happens in plants does not necessarily happen in the same manner in animals.

It is contemplated that the desaturase transgenic animals provided in the present invention are a useful alternate source for the synthesis of LC-PUFAs and can be employed as animal models (including but not limited to models of human disease) in research, including but not limited to pharmaceutical research. In addition, the transgenic animals of the present invention can be used as Bioreactors for the large scale production of LC-PUFAs (See “Molecular Farming: Transgenic Animals as Bioreactors” by J. Van Brunt, Biotechnology, Volume 6, page 1149-1154, 1988, describes the alteration of the genome of various large domestic milk bearing animals yielding transgenic animals capable of producing various heterologous entities. This publication suggests methods for obtaining the primary gene product). The present invention further expands on the above protocols and teaches the the use of transgenic animals for the production of biological products.

Although, there is a considerable body of literature which describes the recombinant or transgenic expression of heterologous glycosyltransferases, the literature does not disclose or in any other manner suggest production of LC-PUFAs in the milk of non-human transgenic mammals as claimed in the present invention.

### DETAILED DESCRIPTION OF PREFERRED EMBODIMENTS AND USES OF THE INVENTION

In preferred embodiments, the present invention has reduced to practice active expression of various desaturase genes (cDNA) in mammalian cells and transgenic animals and provides novel transgenic animals that express specific desaturase genes. The present invention provides transgenic animals expressing desaturases, that have not been described before. In addition, the PUFA derivatives obtained from these transgenic animals, such as the omega-3 products, were not anticipated nor obvious. In yet other embodiments, the transgenic animals of the present invention provides products in animal milk, that were completely unanticipated.

In yet other preferred embodiments, the present invention has reduced to practice, active expression of various desaturase genes (cDNA) into cultured cells and these transformed cultured cells provides products that are not clearly predictable from the available prior art.

In one embodiment, the present invention provides animal cells which express Δ12-desaturase and contains altered levels of linoleic acid, DGLA, AA, adrenic acid, omega-3 PUFAs and their derivatives.

In yet another embodiment, the invention provides a method of modifying levels of linoleic acid, DGLA, AA, adrenic acid, omega-3 PUFAs and their derivatives in animal cells (using Δ12-desaturase alone).

In other embodiments, the milk produced in Δ12-desaturase transgenic animals contains altered levels of linoleic acid, DGLA, AA, adrenic acid, omega-3 PUFAs and their derivatives.

In yet other embodiments, the milk produced in Δ6-desaturase transgenic animals contains altered levels of DGLA, AA, adrenic acid, omega-3 PUFAs and their derivatives.

In other embodiments, the present invention provides a method of modifying levels of linoleic acid, DGLA, AA, adrenic acid, omega-3 PUFAs and their derivatives in transgenic animals (using Δ12-desaturase alone).

In yet other embodiments, the present invention provides a method of modifying levels of DGLA, AA, adrenic acid, omega-3 PUFAs and their derivatives in transgenic animals (using Δ6-desaturase alone).

In another embodiment, the present invention provides animal cells that express Δ6-desaturase which contain altered levels of DGLA, AA, adrenic acid, omega-3 PUFAs and their derivatives (using Δ6-desaturase alone).

In other preferred embodiments the present invention provides animal cells, mammals and milk with altered levels of molecules of the prostaglandin E series. In one embodiment, the present invention provides mammals with altered levels of molecules of the prostaglandin I series, thromboxane series and leukotriene series. In yet another embodiment, the present invention provides a method of altering the levels of molecules of prostaglandin I series, thromboxane series and leukotriene series in mammals.

In other preferred embodiments the present invention provides animal cells, mammals and transgenic animal milk with altered levels of essential fatty acids and omega-3 PUFAs when expressing both Δ12-desaturase and Δ15-desaturase (Δ5-desaturase converts linoleic acid to (α-linolenic acid). In one embodiment, the present invention provides a method of modifying levels of α-linolenic acid and omega-3 PUFAs when expressing both Δ2-desaturase and Δ5-desaturase.

In other preferred embodiments the present invention provides a method of producing animal cells or mammals which do not require external supply of essential fatty acids.

### USES OF THE INVENTION

A. The following broad applications or uses of the present invention are contemplated:

1. Fat Free Media: Growth of tissue culture cells in medium lacking essential fatty acids, especially linoleic acid. This allows individuals and companies to better define media used for maintaining cultured vertebrate cells. It may also improve the ability of cultured cells to produce recombinant proteins and/or make the process more economical.

2. Research Reagent: Since obtaining essential fatty acids and their derivatives, and long chain poly-unsaturated fatty acids is expensive, these molecule by and large have not been used in the laboratory. The ability to generate these molecule would open a market for their use in countless numbers of research laboratories.

B. Methods of Treatment and Formulations

In particular, the present invention provides the following methods and/or products which are useful for the applications as mentioned:

1. Method of treating or preventing malnutrition by administering milk fat or animal fat, or fraction thereof, in an amount sufficient to affect the treatment or prevention.

2. Method of treating patients which exhibit condition(s) caused by either inadequate intake or inadequate endogenous production of PUFAs. Treatment would be administration of dietary substitute or supplements containing the milk/animal fats, or fraction thereof, produced in the desaturase expression systems.

3. A pharmaceutical composition comprising the milk fat or animal fat, or fraction thereof.

4. A nutritional formula comprising the milk fat or animal fat, or fraction thereof. The nutritional formula comprise of and include infant formula, dietary supplements and dietary substitutes and, where applicable, administratable to both humans and animals.

5. Cosmetics comprising the milk fat or animal fat, or fraction thereof

6. Animal feeds comprising the milk fat or animal fat, or fraction thereof.

### EXPERIMENTAL

In the experimental disclosure which follows, the following abbreviations and methodology apply: g (gram); mg (milligrams); μg (microgram); M (molar); mM (milliMolar); μM (microMolar); nm (nanometers); L (liter); ml (milliliter); μl (microliters);° C. (degrees Centigrade); m (meter); sec. (second); DNA (deoxyribonucleic acid); cDNA (complementary DNA); RNA (ribonucleic acid); mRNA (messenger ribonucleic acid); PAGE (polyacrylamide gel electrophoresis); BAP (6-benzyl aminopurine); Tris (tris (hydroxymethyl) -aminomethane); PBS (phosphate buffered saline); 2×SSC (0.3 M NaCl, 0.03 M Na₃citrate, pH 7.0); Gibco BRL (Gaithersburg, Md.); Sigma (St. Louis, Mo.).

Methodology

Cell Culture And Generation Of Stable Cell Lines: Mouse L cells (ATCC)[thymidine kinase negative (TK⁻) and adenine phosphoribosyltransferase negative (APRT⁻)] were maintained in Dulbecco's modified Eagle's medium (DMEM, Gibco Laboratories, Grand Island, N.Y.) containing 10% Nu-serum (Collaborative Research Inc., Bedford, Mass.), 50 ug/ml gentamicin sulfate (Gibco) and 50 ug/ml 2,6-di-aminopurine (Sigma, St. Louis, Mo.).

Stably transfected L cells were generated using the calcium phosphate precipitation method as previously described [M. Wigler et al., “Transfer of purified herpes virus thymidine kinase gene to cultured mouse cells,” Cell 11:223-232 (1977); A. Pellicer et al., “Altering genotype and phenotype by DNA-mediated gene transfer,” Science 209:1414-1422 (1980); B. Wold et al., “Introduction and expression of a rabbit b-globin gene in mouse fibroblasts,” roc. Natl. Acad. Sci. USA 76:5684-5688 (1979); J. M. Roberts and R. Axel, “Gene amplification and gene correction in somatic cells,” Cell 29:109-119 (1982)]. This method utilizes a plasmid DNA (pDIAT3) which encodes two selectable markers: an intact APRT gene and a truncated TK gene lacking transcriptional regulatory (enhancer) sequences. Cells were first selected for the APRT⁺ phenotype in DMEM containing 10% Nu-serum, 4 ug/ml azaserine, 15 ug/ml adenine and 50 ug/ml gentamicin sulfate. The APRT⁺ colonies were then grown in DMEM containing 10% Nu-serum, 50 ug/ml gentamicin sulfate, 15 ug/ml hypoxanthine, 1 ug/ml aminopterin and 5.15 ug/ml thymidine (HAT medium) to select for the TK⁺ phenotype. Individual TK⁺ clones were isolated from the HAT⁺ pool by limited dilution of the cells into 96-well plates at a concentration of 0.5 cells/well. Individual TK³⁰ clones were analyzed for the presence of integrated desaturase sequences by slot blot hybridization analysis using a [³²P]radiolabeled DNA probe containing sequences from bGH exon V and 3′ untranslated region. TK⁺ clones containing integrated CMVie-Δ6-bGH or pCMVie-Δ12-bGH sequences were expanded and analyzed further.

Stably transfected Hela cells were generated using the Lipofectamine Reagent method (BRL, Gaithersburg, Md.) as suggested by the manufacturer. Briefly, 300 ng of plasmid DNA encoding the neomycin phosphotransferase gene driven by the RSV-LTR TRE [C. M. Gorman et al., “The Rous sarcoma virus long terminal repeat is a strong promoter when introduced into a variety of eukaryotic cells by DNA-mediated transfection,” Proc. Natl. Acad. Sci. USA 79:6777-6781 (1982a)] and 30 ug of pCMVie-Δ12-bGH plasmid DNA was diluted in H₂O to a volume of 200 ul. This was added to 200 ul of a 25% Lipofectamine solution, mixed gently and allowed to stand for 30 min at room temperature. Approximately 1×10⁶ Hela cells in a 100 mm dish were washed twice with 10ml serum-free DMEM and 4 ml serum-free DMEM was added. The above DNA/Lipofectamine solution was then added to the cells and incubated for 5 hr at 37°. Following incubation, the medium containing the DNA/Lipofectamine was removed and replaced with culture medium. After 24 hr, the cells were passed into culture medium containing 300 ug/ml G-418 sulfate (Geneticin, Gibco) to select for cells exhibiting neomycin resistance. The neomycin resistant Hela cell pool was then expanded and analyzed further.

The following examples serve to illustrate certain preferred embodiments and aspects of the present invention and are not to be construed as limiting the scope thereof

### EXAMPLE 1

This example describes the construction of the eukaryotic desaturase expression vectors. DNA manipulations were carried out using standard cloning techniques. A 1,382 bp EcoRI-XhoI DNA fragment encoding the Δ6-desaturase gene was isolated from plasmid pCGR5 and ligated into plasmid pCMV-BGH-C [A. Martin-Gallardo et al., “A comparison of bGH expression in mouse L cells directed by the Moloney murine leukemia virus long terminal repeat, the simian virus 40 early or cytomegalovirus immediate early promotors,” Gene 70:151-156 (1988)], which had been cleaved with BglII and SmaI. The termini of the DNA molecules were made flush using Klenow polymerase prior to ligation. The resulting plasmid, pCMVie-Δ6-bGH, utilizes the cytomegalovirus immediate early transcriptional regulatory element to direct A-6-desaturase transcription and the bGH polyadenylation signal for proper processing of the 3′ terminus of desaturase mRNA (See FIG. 2A). Similarly, a 1,209 bp EcoRI-XhoI DNA fragment encoding the Δ12-desaturase gene was isolated from plasmid pCGR7 and ligated into plasmid pCMV-BGH-C which had been cleaved with BglII and SmaI to generate the plasmid, pCMVie-Δ12-bGH (See FIG. 2B). The termini of these DNA molecules were also made flush using Klenow polymerase prior to ligation.

The DNA fragments encoding the Δ6-desaturase gene and the Δ12-desaturase gene were also ligated into plasmid, pWAP-polyA [Prieto et al.(1995)] which had been cleaved with SmaI (See FIGS. 2C and 2D). The resulting plasmids, pWap-Δ6-bGH and pWap-Δ12-bGH utilize the murine whey acidic protein transcriptional regulatory element to direct Δ6 and Δ12-desaturase transcription and the bGH polyadenylation signal for proper processing of the 3′ terminus of desaturase mRNAs. The murine whey acidic protein transcriptional regulatory element has been shown to direct gene expression primarily to lactating mammary gland tissue [C. W. Pittuis et al., Proc. Natl. Acad. Sci. U.S.A. 85:5874-5878 (1988)].

### EXAMPLE 2

This example describes the eneration of transgenic animals, expressing the desaturase genes.

Plasmid pWap-Δ6-bGH was cleaved with restriction endonucleases EcoRI and PstI. A linear DNA fragment containing sequences encoding the WAP-Δ6-bGH transcriptional unit was isolated and injected into fertilized mouse (B6/SJL) eggs as described previously [M. M. McGrane et al., J. Biol. Chem. 263:11443-11451 (1988)]. The injected eggs were transferred to pseudopregnant females which subsequently delivered pups. High molecular weight chromosomal DNA was isolated from tail biopsies of the pups and was analyzed for the presence of integrated transgene sequences by slot blot hybridization analysis using a [³²P]radiolabeled DNA probe containing sequences from bGH exon V and 3′ untranslated region. Similarly, plasmid pWap-Δ12-bGH was cleaved with restriction endonucleases EcoRI and BamHI. A linear DNA fragment containing sequences encoding the WAP-Δ12-bGH transcriptional unit was isolated, injected and transgenic animals identified. Control, Δ6 and Δ12 females were mated and allowed to deliver F₁ progeny. Milk samples were obtained from control and transgenic mothers 5-12 days postpartum and then analyzed for desaturase activity.

### EXAMPLE 3

In this example, experiments have been described that show expression of Δ6 and Δ12-desaturases in cell culturein vitro. Nine stably transfected L cell clones containing integrated Δ12-desaturase sequences (Δ12) were obtained as described in the methodology. T-25cm² flasks of control (L) and Δ12 cells were incubated in serum-containing medium or in serum-free medium for either one or three days. This incubation deprived the cells of essential fatty acids (linoleic acid, 18:2n-6 and α-linolenic acid, 18:3n3). Following incubation, the cells were isolated and analyzed for Δ12-desaturase activity by determining the cellular levels of various omega-6 fatty acids as a percentage of total fatty acid. The results are summarized in Table 1. The values given for Δ12-desaturase L cell clones represents the mean values for the nine clones analyzed.

TABLE 1

| | | | | Δ12-desaturase L | | |
|---|---|---|---|---|---|---|
| | Control L Cells | | | Cell Clones (N = 9) | | |
| | | Serum-Free Medium | | Serum-Free Medium | | |
| Fatty Acid | +Serum | 1 | 3 | +Serum | 1 | 3 (Days) |
| 18:2n-6 | 3.25 | 1.77 | 1.13 | 19.99 | 21.52 | 24.04 |
| 18:3n-6 | 0.10 | nd* | 0.16 | 0.15 | 0.16 | 0.15 |
| 20:2n-6 | 0.13 | 0.17 | 0.12 | 0.67 | 1.09 | 1.48 |
| 20:3n-6 | 0.20 | 0.44 | 0.19 | 1.20 | 1.21 | 0.70 |
| 20:4n-6 | 0.58 | 1.72 | 1.04 | 3.53 | 4.06 | 2.98 |
| 22:4n-6 | 0.05 | 0.21 | 0.15 | 0.26 | 0.32 | 0.31 |

*nd = not detected (<0.05).

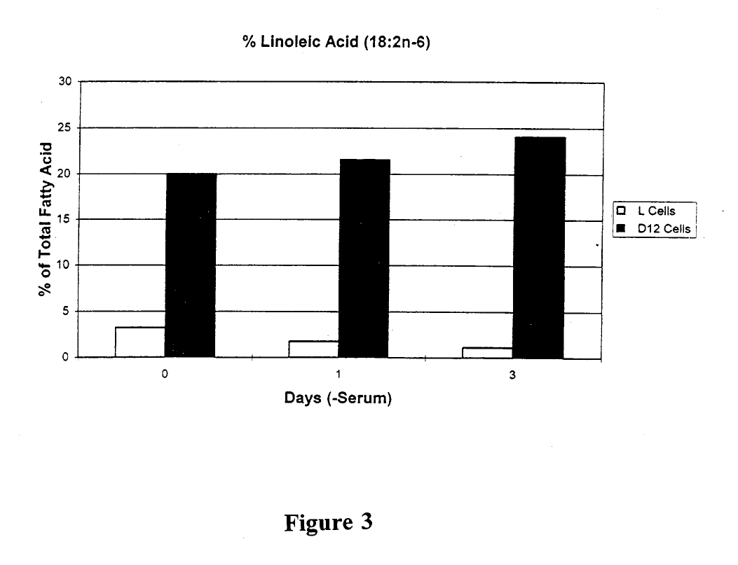

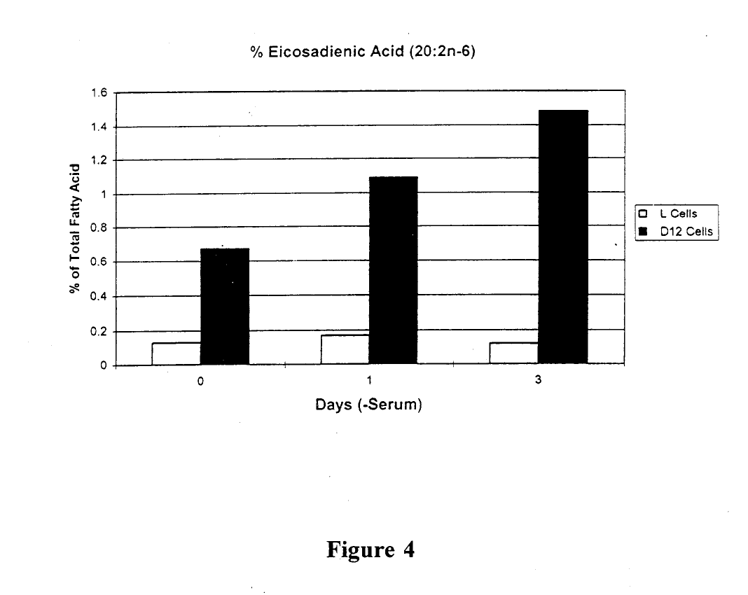

In medium containing serum, linoleic acid (18:2n-6) comprised approximately 3.25% of total fatty acid in control cells. The percentage of this fatty acid in control cells decreased from 3.25 to 1.77 and 1.13 when the cells were incubated in serum-free medium for 1 and 3 days, respectively. In contrast, Δ12 cells had significantly elevated levels of linoleic acid. In medium containing serum, linoleic acid comprised approximately 19.99% of total fatty acid in Δ12 cells. This represented a 515% increase in Δ12 cells compared to control cells. The levels of linoleic acid increased to 21.52 and 24.04% when the cells were incubated cells in serum-free medium for 1 and 3 days, respectively (See FIG. 3). The 24.04% level of linoleic acid in Δ12 cells in serum-free medium for 3 days represented a 2,027% increase from what was present in control cells treated similarly. These results demonstrated that the Δ12-desaturase was expressed in these cells and converted oleic acid (18:1n-9) to linoleic acid (18:2n-6). Analysis of other omega-6 fatty acids from cultures incubated in serum-free medium for 24 hours indicates: 1) γ-linolenic acid (GLA, 18:3n-6) was elevated in Δ12 cells compared to control cells, 0.16 verses undetectable (<0.05); and 2) di-homo-γ-linolenic acid (20:3n-6) and arachidonic acid (20:4n-6) were both significantly elevated in Δ12 cells compared to control cells, 1.21 and 4.06% verses 0.44 and 1.72%, respectively. The percentage of another fatty acid, eicosadienic acid (20:2n-6), in control cells was approximately 0.13% in serum-containing medium and remained relatively constant (0.17 and 0.12%) when the cells were incubated cells in serum-free medium for 1 and 3 days, respectively. In contrast, 20:2n-6 levels in Δ12 cells increased from 0.67% in serum-containing medium to 1.09 and 1.48% when the cells were incubated cells in serum-free medium for 1 and 3 days, respectively (See FIG. 4).

Analysis of omega-3 fatty acids indicated that both eicosapentaenoic acid (20:5n-3) and docosapentaenoic acid (22:5n-3) were significantly elevated in Δ12 cells compared to control cells. The level of docosahexaeonic acid (22:6n-3) was also elevated in Δ12 cells compared to control cells but the increase was less pronounced (less than a 100% increase). The levels (% of total fatty acid) of various fatty acids in serum-containing and serum-free medium are summarized in Table 2.

TABLE 2

| | | | | Δ12-desaturase L | | |
|---|---|---|---|---|---|---|
| | Control L Cells | | | Cell Clones (N = 9) | | |
| | | Serum-Free Medium | | Serum-Free Medium | | |
| Fatty Acid | +Serum | 1 | 3 | +Serum | 1 | 3 (Days) |
| 18:3n-3 | 0.13 | 0.36 | 0.30 | 1.24 | 0.54 | 0.26 |
| 18:4n-3 | 0.07 | nd* | 0.17 | 0.13 | 0.12 | 0.18 |
| 20:4n-3 | 0.04 | nd | nd | 0.37 | 0.36 | 0.33 |
| 20:5n-3 | 0.03 | 0.77 | 0.34 | 2.34 | 1.84 | 1.06 |
| 22:5n-3 | 0.08 | 0.82 | 0.38 | 4.79 | 5.48 | 4.37 |
| 22:6n-3 | 0.17 | 0.45 | 0.27 | 0.75 | 0.75 | 0.53 |

*nd = not detected (<0.05).

Stably transfected Hela cells containing integrated Δ12-desaturase sequences (Δ12) were generated as described previously. Increased Δ12-desaturase activity in this human epithelial cell line was important to demonstrate that the enzyme was capable of altering the fatty acid profile in multiple species and cell types. Control Hela and Δ12-Hela cells were isolated from cultures growing in serum-containing medium as well as duplicate cultures incubated for 24 hours in serum-free medium. Table 3 summarizes the levels of various omega-6 and omega-3 fatty acids in these cells as a percentage of total fatty acid.

The Δ12-Hela cells had slightly elevated levels of the Δ12-desaturase product, linoleic acid (18:2n-6) compared to control Hela cells. However, the levels of both arachidonic acid (20:4n-6) and adrenic acid (22:4n-6) were significantly elevated in the Δ12-Hela cells. Similarly, the levels of both docosapentaenoic acid (22:5n-3) and docosahexaeonic acid (22:6n-3) were elevated in the omega-3 pathway of Δ12-Hela cells compared to control Hela cells. These results demonstrated that the Δ12-desaturase was expressed in these Hela cells and converted oleic acid (18:1n-9) to linoleic acid (18:2n-6). The results also indicated that Hela cells had highly active endogenous Δ6-desaturase, elongase and Δ5-desaturase enzymes. These enzymes rapidly elongated and desaturated the increased pool of linoleic acid though the omega-6 (and omega-3) pathway to generate elevated levels of arachidonic acid (20:4n-6) and other fatty acids.

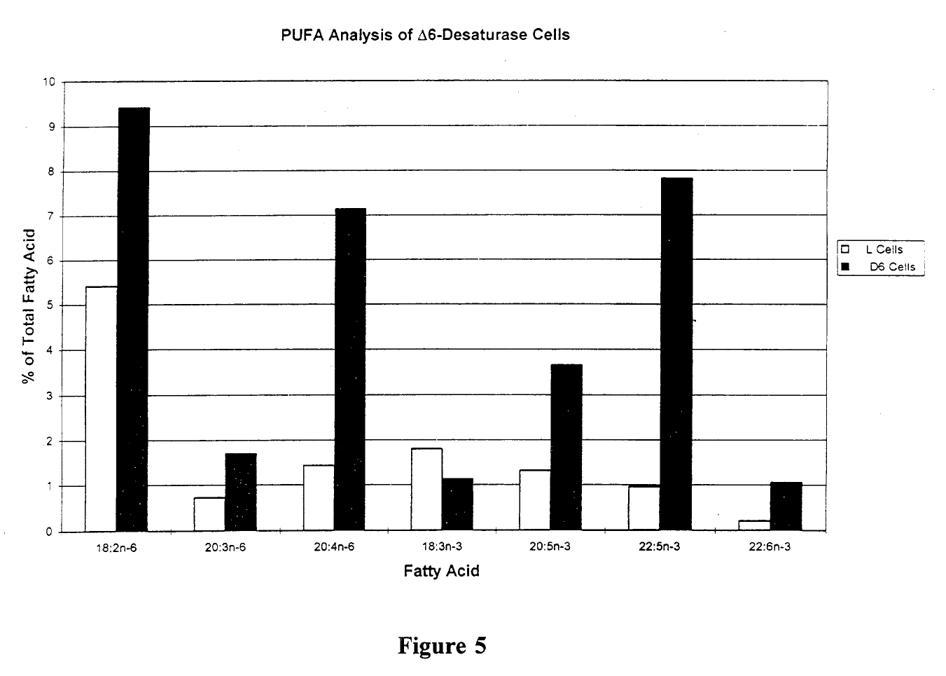

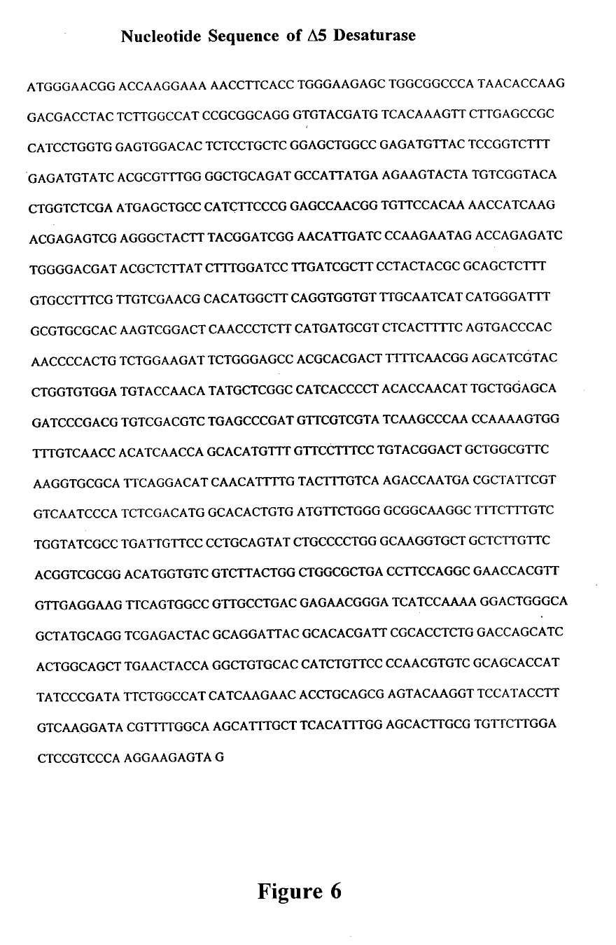

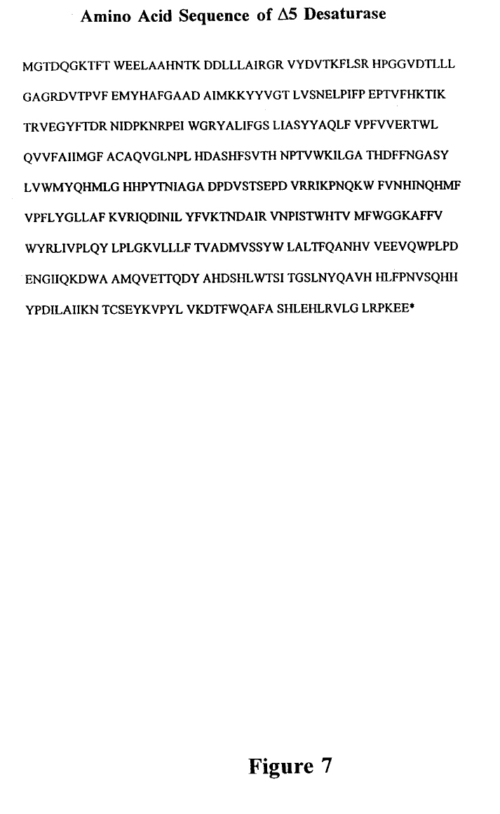

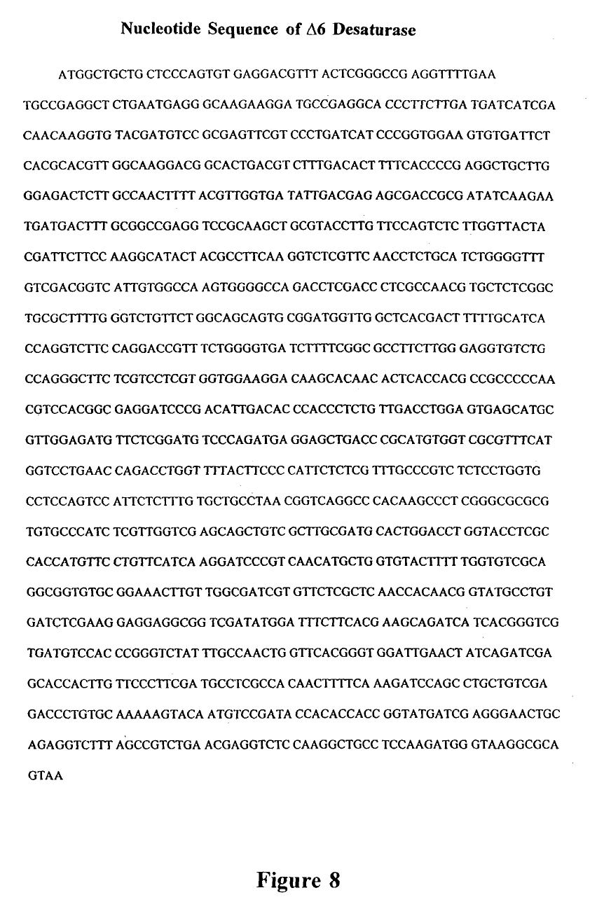

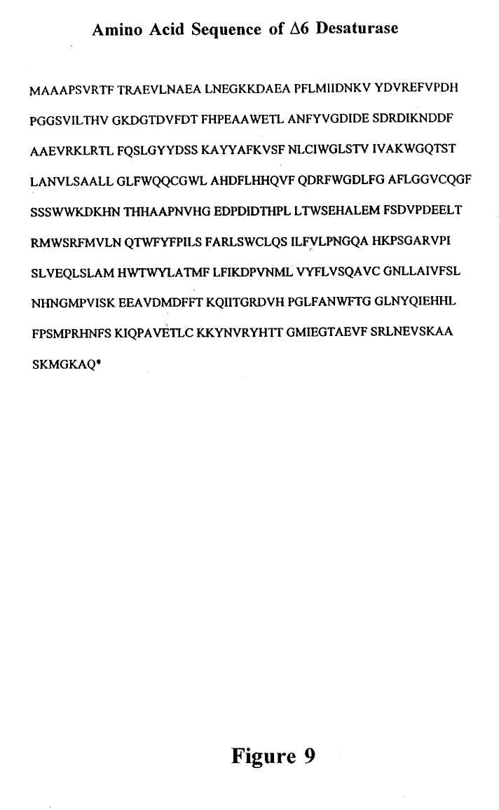

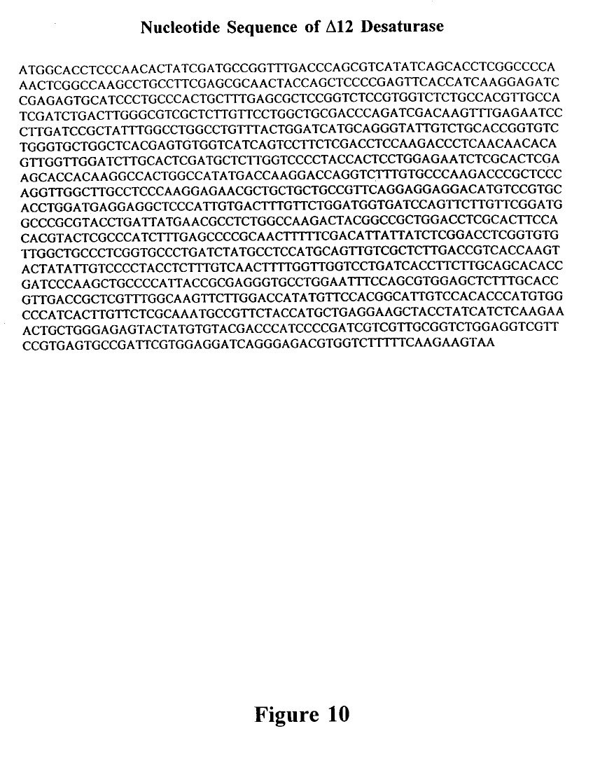

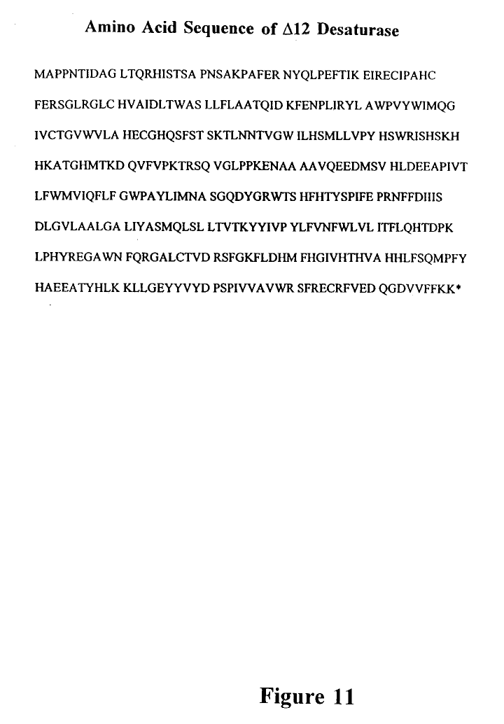

Seven stably transfected L cell clones containing integrated Δ6-desaturase sequences (Δ6) were obtained as described. Since L cells expressing Δ6-desaturase would be expected to still require essential fatty acids, control (L) and Δ6 cells were isolated from growing cultures (serum-containing culture medium). The cells were analyzed for Δ6-desaturase activity by determining the levels of various omega-6 and omega-3 fatty acids within the cells as a percentage of total fatty acid. The results are summarized in Table 4 and in (FIG. 5). The values given for Δ6-desaturase L cell clones represent the mean values for the seven clones analyzed.

TABLE 3

| Medium | |
|---|---|
| (+)Serum | (−)Serum |

| Fatty Acid | Hela | Δ12-Hela | Hela | Δ12-Hela |
|---|---|---|---|---|
| 18:2n-6 | 1.85 | 2.59 | 1.74 | 2.21 |
| 20:2n-6 | nd* | 0.34 | 1.13 | nd |
| 20:3n-6 | nd | 0.75 | 0.43 | nd |
| 20:4n-6 | 5.03 | 12.07 | 9.22 | 12.53 |
| 22:4n-6 | 1.53 | 2.63 | 1.88 | 3.35 |
| 18:3n-3 | nd | nd | nd | nd |
| 20:5n-3 | nd | 1.01 | 0.55 | nd |
| 22:5n-3 | nd | 10.14 | 2.57 | 3.49 |
| 22:6n-3 | nd | 3.92 | 3.42 | 3.34 |

*nd = not detected (<0.05).

TABLE 4

| | | Δ6-desaturase |
|---|---|---|
| Fatty Acid | L Cells (−C) | L Cell Clones (N = 7) |
| 18:2n-6 | 5.40 | 9.42 |
| 18:3n-6 | nd* | nd |
| 20:2n-6 | 0.19 | 0.26 |
| 20:3n-6 | 0.73 | 1.70 |
| 20:4n-6 | 1.43 | 7.14 |
| 18:3n-3 | 1.80 | 1.12 |
| 18:4 | nd | nd |
| 20:4n-3 | nd | nd |
| 20:5n-3 | 1.31 | 3.66 |
| 22:5n-3 | 0.96 | 7.82 |
| 22:6n-3 | 0.21 | 1.06 |

* nd = not detected (<0.05).

All L cell clones containing stably integrated Δ6-desaturase sequences contained significantly elevated levels of di-homo-γ-linolenic acid (20:3n-6) and arachidonic acid (20:4n-6) compared to control L cells, with the mean increases being 133% and 399%, respectively. In addition, the levels of these fatty acids in the Δ6-desaturase clones were also elevated compared to L cells expressing the Δ12-desaturase. In the omega-3 series, there was a 38% decrease in the level of α-linolenic acid (18:3n3) in the Δ6-desaturase cells compared to control cells. However, the levels of eicosapentaenoic acid (20:5n-3), docosapentaenoic acid (22:5n-3) and docosahexaenoic acid (22:6n-3) were all elevated in the Δ6-desaturase cells compared to both control and Δ12-desaturase cells. The increases above control cells for these fatty acids were 179%, 715% and 405%, respectively. These data demonstrated that the Δ6-desaturase was expressed in these cells and converted linoleic acid (18:2n-6) to γ-linolenic acid (18:3n-6) and α-linolenic acid (18:3n3) to steridonic acid (18:4n-3). The γ-linolenic acid (18:3n-6) was then converted to di-homo γ-linolenic acid (20:3n-6) by elongase whereas the steridonic acid (18:4n-3) was converted to eicosatrienoic acid (20:4n-3) by elongase and further desaturated to eicosapentaenoic acid (20:5n-3) and docosahexaeonic acid (22:6n-3) by the endogenous Δ5 and Δ4-desaturases.

### EXAMPLE 4

In this example, experiments have been described that show expression of Δ6 and Δ12-desaturase transgenes in mice.

Female mice containing either the Δ6 and Δ12-desaturase transgene were generated as described previously in the Methodology. The expression these transgenes is directed by the murine whey acidic protein promoter and expected to be restricted mainly to the lactating mammary gland. The females were mated and milk was collected from each lactating mother 5-12 days post partum. The milk was analyzed for Δ6 and Δ12-desaturase activity by determining the levels of various omega-6 and omega-3 fatty acids present within the milk, as a percentage of total fatty acid, compared to those found in control mouse milk. The results are summarized in Table 5.

TABLE 5

| | Nontransgenic | Transgenic Mouse Milk | | | |
|---|---|---|---|---|---|
| Fatty Acid | Milk | Δ6 F₀58 | Δ12 F₀13 | Δ12 F₀20 | Δ12 F₀76 |
| 18:2n-6 | 13.29 | 14.18 | 13.85 | 12.04 | 14.92 |
| 18:3n-6 | 0.07 | 0.22 | 0.13 | 0.17 | 0.18 |
| 20:3n-6 | 0.49 | 0.74 | 0.54 | 0.62 | 0.80 |
| 20:4n-6 | 0.34 | 0.61 | 0.51 | 0.54 | 0.72 |
| 22:4n-6 | 0.17 | 0.19 | 0.26 | 0.24 | 0.31 |
| 18:3n-3 | 0.93 | 0.89 | 0.94 | 0.82 | 0.90 |
| 18:4 | 0.17 | 0.09 | 0.19 | 0.19 | 0.21 |
| 20:3n-3 | 0.38 | 0.22 | nd* | nd | nd |
| 20:4n-3 | 0.13 | 0.14 | 0.12 | 0.14 | 0.17 |
| 20:5n-3 | 0.22 | 0.39 | 0.26 | 0.33 | 0.40 |
| 22:5n-3 | 0.44 | 0.44 | 0.56 | 0.51 | 0.63 |
| 22:6n-3 | 0.54 | 0.53 | 0.54 | 0.52 | 0.75 |

F₀ = Founder line.
*nd = not detected (<0.05).

Analysis of omega-6 fatty acids from Δ6 F₀58 milk indicated that γ-linolenic acid (18:3n-6), di-homo-γ-linolenic acid (20:3n-6) and arachidonic acid (20:4n-6) were all significantly increased compared to control milk (0.22, 0.74 and 0.61% versus 0.07, 0.49 and 0.34%, respectively). Analysis of omega-3 fatty acids from the Δ6 F₀58 milk indicated that eicosapentaenoic Acid (20:5n-3) was significantly elevated in the transgenic milk compared control milk (0.39 versus 0.22%). Similar increases were observed in milk samples obtained from Δ12 transgenic animals. The transgenic line, Δ12 F₀76, demonstrated the largest increases in γ-linolenic acid (18:3n-6), di-homo γ-linolenic acid (20:3n-6) and arachidonic acid (20:4n-6) compared to control milk (0.18, 0.80 and 0.72% compared to 0.07, 0.49 and 0.34%, respectively). Milk from this animal also demonstrated significant elevations in several omega-3 fatty acids. While the levels of 60 -linolenic acid (18:3n3), steridonic acid (18:4n-3) and eicosatrienoic acid (20:4n-3) were not elevated, significant increases were found in the levels of eicosapentaenoic acid (20:5n-3), docosapentaenoic acid (22:5n-3) and docosahexaenoic acid (22:6n-3). The levels of these fatty acids compared to those found in control milk was 0.40, 0.63 and 0.75% versus 0.22, 0.44 and 0.54, respectively. These results demonstrated that transgenic mouse lines were generated which express the Δ6-desaturase and Δ12-desaturase genes. The expression of these genes in the mammary gland of the lactating animal resulted in increased levels of several omega-3 and omega-6 fatty acids compared to those found in nontransgenic mouse milk.

The above examples describe novel results obtained in cultured animal cells and transgenic animals, which were unexpected, and have not been described previously.

We claim:

1. A composition, comprising animal cells producing essential fatty acids.

2. The composition of claim 1, wherein said animal cells comprise nucleic acid encoding a heterologous desaturase.

3. The composition of claim 1, wherein said nucleic acid comprises a heterologous desaturase gene selected from the group consisting of Δ5-desaturase, Δ6-desaturase, Δ12-desaturase, and Δ15-desaturase genes.

4. The composition of claim 3, wherein said animal cells are mammalian cells.

5. The composition of claim 4, wherein said animal cells produce arachidonic acid.

6. The composition of claim 4, wherein said mammalian cells are in tissue culture.

7. The composition of claim 4, wherein said mammalian cells are in a bioreactor.

8. A nutritional formulation, comprising at least one essential fatty acid derived from the animal cells of claim 1.

9. An animal feed formulation, comprising at least one essential fatty acid derived from the animal cells of claim 1.

10. A composition, comprising transfected animal cells, said transfected animal cells producing altered levels of long-chain polyunsaturated fatty acids relative to the animal cells prior to transfection.

11. The composition of claim 10, wherein said animal cells comprise nucleic acid encoding a heterologous desaturase.

12. The composition of claim 11, wherein said nucleic acid comprises a heterologous desaturase gene selected from the group consisting of Δ5-desaturase, Δ6-desaturase, Δ12-desaturase, and Δ15-desaturase genes.

13. The composition of claim 10, wherein said animal cells are mammalian cells.

14. The composition of claim 13, wherein said mammalian cells are in tissue culture.

15. The composition of claim 13, wherein said mammalian cells are in a bioreactor.

16. A nutritional formulation, comprising at least one long-chain polyunsaturated fatty acid derived from the animal cells of claim 10.

17. A cosmetic formulation, comprising at least one long-chain polyunsaturated fatty acid derived from the animal cells of claim 10.

18. An animal feed formulation, comprising at least one long-chain polyunsaturated fatty acid derived from the animal cells of claim 10.

19. A method, comprising: a) providing: i) a non-human animal cell, ii) a vector comprising nucleic acid encoding a heterologous desaturase, and iii) a recipient non-human female animal; b) introducing said vector into said cell to create a transfected cell; and c) transferring said transfected cell into said recipient female under conditions such that at least one offspring is produced, said offspring expressing said desaturase in one or more tissues.

20. The method of claim 19, wherein said offspring expresses said desaturase in said offspring's mammary tissue, resulting in the production of essential fatty acids in the offspring's milk.

21. The method of claim 20, further comprising step d) collecting said milk.

22. The method of claim 21, further comprising step e) isolating said essential fatty acids in said milk.

23. The method of claim 22, further comprising step (f) synthesizing one or more downstream products from said essential fatty acids islated in step (e).

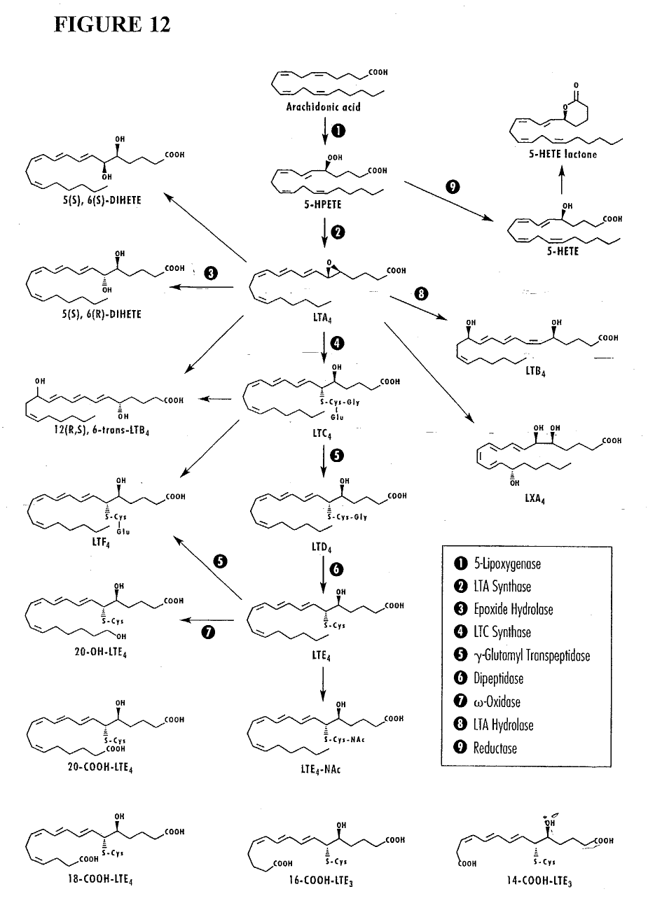

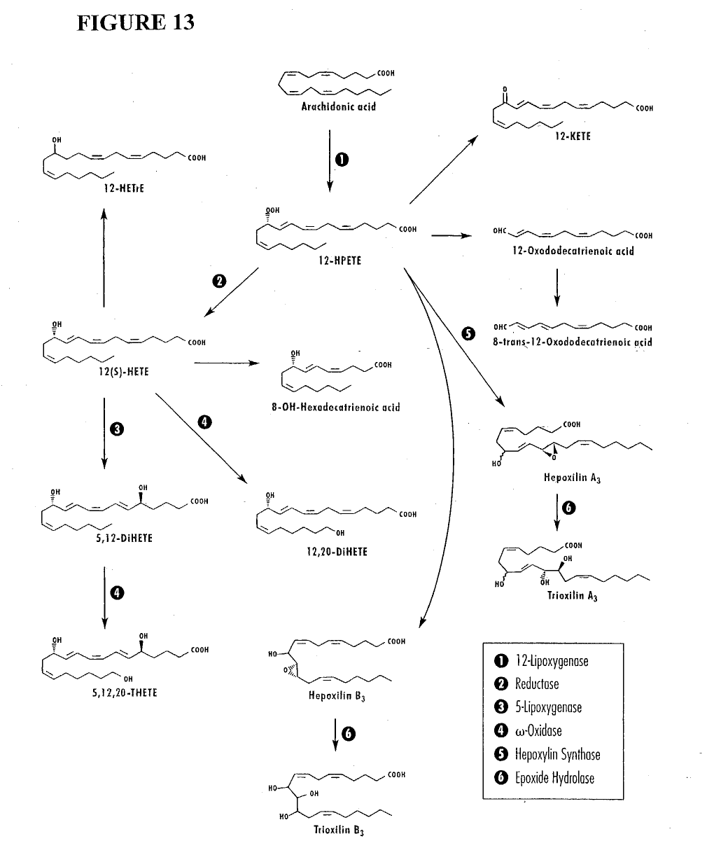

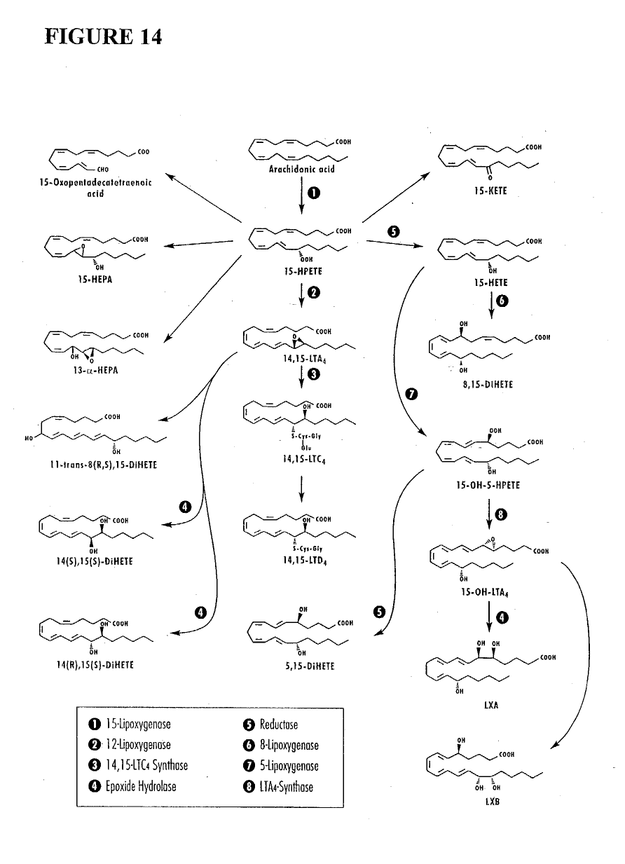

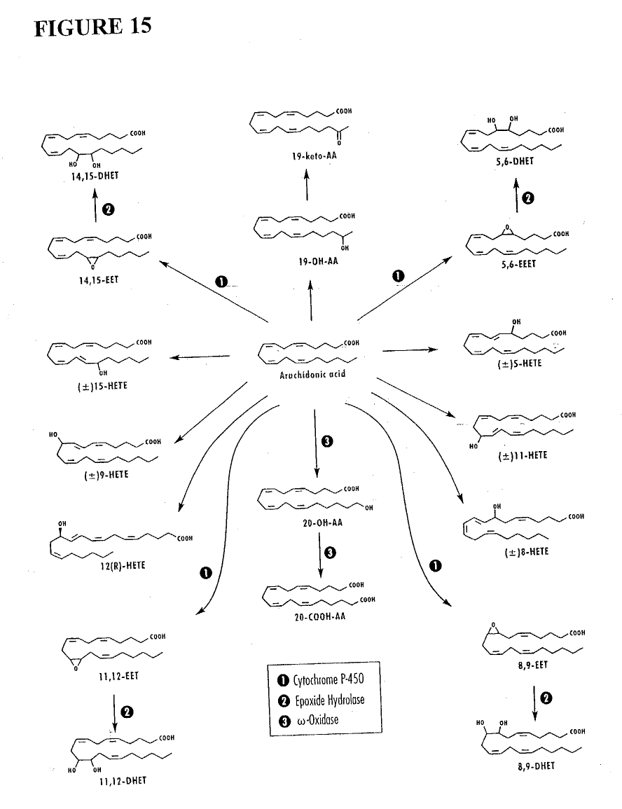

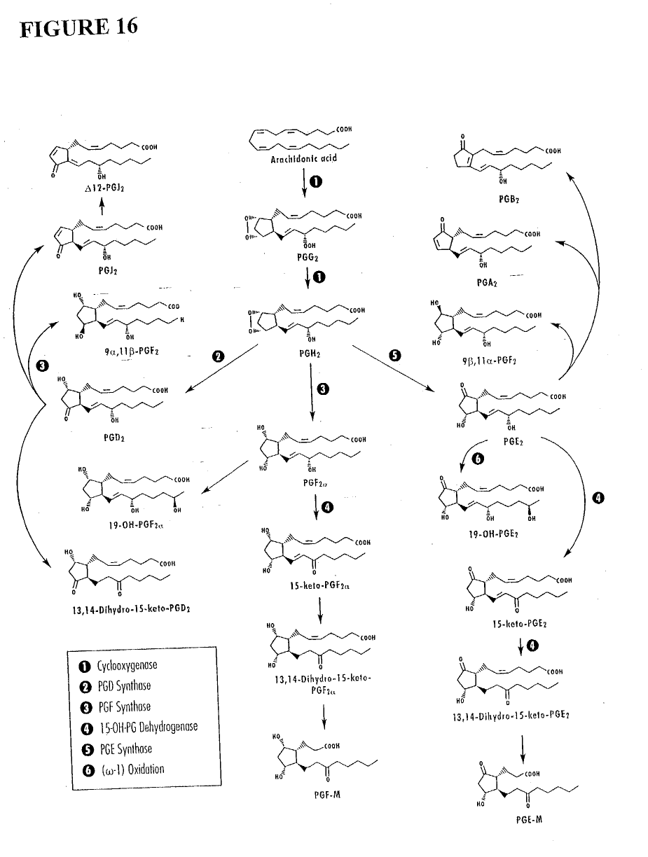

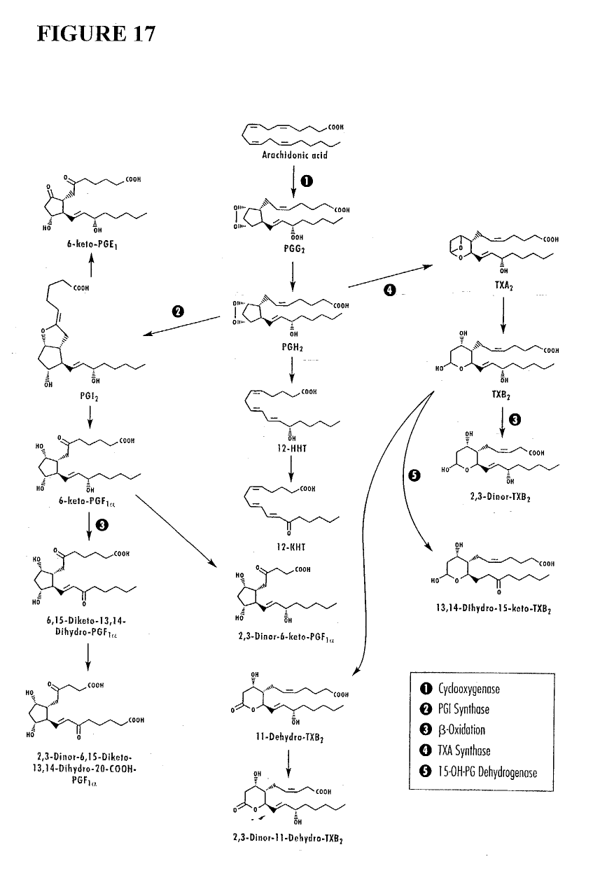

24. The method of claim 23, wherein said one or more downstream products is selected from the group consisting of those downstream products set forth in FIGS. 12, 13, 14, 15, 16 and 17.

25. The method of claim 23, wherein said one or more downstream products is selected from the group consisting of leukotrienes and thromboxanes.

26. The method of claim 19, wherein said nucleic acid comprises a desaturase gene selected from the group consisting of Δ5-desaturase, Δ6-desaturase, Δ12-desaturase, and Δ5-desaturase genes.

27. The method of claim 26, wherein said desaturase gene is obtained from a fungal source.

28. The method of claim 26, wherein said desaturase gene is obtained from a plant source.

29. The method of claim 19, wherein said non-human female is selected from the group consisting of mice, rats, rabbits, pigs, goats, sheep, cows and horses.

30. A composition, comprising the milk of the offspring produced according to the method of claim 20.

31. A transgenic non-human animal, said animal producing at least one product, said product selected from the group consisting of arachidonic acid, eicosapentaenoic acid, docosahexaenoic acid.


SEQUENCE LISTING (6 sequences)

SEQ ID NO: 1 - DNA (other nucleic acid), 1341 bp; double-stranded, linear
  description: /desc = “DNA”
atgggaacggaccaaggaaaaaccttcacctgggaagagctggcggcccataacaccaaggacgacctactcttggccatccgcggcagggtgtacgatgtcacaaagttcttgagccgccatcctggtggagtggacactctcctgctcggagctggccgagatgttactccggtctttgagatgtatcacgcgtttggggctgcagatgccattatgaagaagtactatgtcggtacactggtctcgaatgagctgcccatcttcccggagccaacggtgttccacaaaaccatcaagacgagagtcgagggctactttacggatcggaacattgatcccaagaatagaccagagatctggggacgatacgctcttatctttggatccttgatcgcttcctactacgcgcagctctttgtgcctttcgttgtcgaacgcacatggcttcaggtggtgtttgcaatcatcatgggatttgcgtgcgcacaagtcggactcaaccctcttcatgatgcgtctcacttttcagtgacccacaaccccactgtctggaagattctgggagccacgcacgactttttcaacggagcatcgtacctggtgtggatgtaccaacatatgctcggccatcacccctacaccaacattgctggagcagatcccgacgtgtcgacgtctgagcccgatgttcgtcgtatcaagcccaaccaaaagtggtttgtcaaccacatcaaccagcacatgtttgttcctttcctgtacggactgctggcgttcaaggtgcgcattcaggacatcaacattttgtactttgtcaagaccaatgacgctattcgtgtcaatcccatctcgacatggcacactgtgatgttctggggcggcaaggctttctttgtctggtatcgcctgattgttcccctgcagtatctgcccctgggcaaggtgctgctcttgttcacggtcgcggacatggtgtcgtcttactggctggcgctgaccttccaggcgaaccacgttgttgaggaagttcagtggccgttgcctgacgagaacgggatcatccaaaaggactgggcagctatgcaggtcgagactacgcaggattacgcacacgattcgcacctctggaccagcatcactggcagcttgaactaccaggctgtgcaccatctgttccccaacgtgtcgcagcaccattatcccgatattctggccatcatcaagaacacctgcagcgagtacaaggttccataccttgtcaaggatacgttttggcaagcatttgcttcacatttggagcacttgcgtgttcttggactccgtcccaaggaagagtag

SEQ ID NO: 2 - protein, 446 aa
MGTDQGKTFTWEELAAHNTKDDLLLAIRGRVYDVTKFLSRHPGGVDTLLLGAGRDVTPVFEMYHAFGAADAIMKKYYVGTLVSNELPIFPEPTVFHKTIKTRVEGYFTDRNIDPKNRPEIWGRYALIFGSLIASYYAQLFVPFVVERTWLQVVFAIIMGFACAQVGLNPLHDASHFSVTHNPTVWKILGATHDFFNGASYLVWMYQHMLGHHPYTNIAGADPDVSTSEPDVRRIKPNQKWFVNHINQHMFVPFLYGLLAFKVRIQDINILYFVKTNDAIRVNPISTWHTVMFWGGKAFFVWYRLIVPLQYLPLGKVLLLFTVADMVSSYWLALTFQANHVVEEVQWPLPDENGIIQKDWAAMQVETTQDYAHDSHLWTSITGSLNYQAVHHLFPNVSQHHYPDILAIIKNTCSEYKVPYLVKDTFWQAFASHLEHLRVLGLRPKEE

SEQ ID NO: 3 - DNA (other nucleic acid), 1374 bp; double-stranded, linear
  description: /desc = “DNA”
atggctgctgctcccagtgtgaggacgtttactcgggccgaggttttgaatgccgaggctctgaatgagggcaagaaggatgccgaggcacccttcttgatgatcatcgacaacaaggtgtacgatgtccgcgagttcgtccctgatcatcccggtggaagtgtgattctcacgcacgttggcaaggacggcactgacgtctttgacacttttcaccccgaggctgcttgggagactcttgccaacttttacgttggtgatattgacgagagcgaccgcgatatcaagaatgatgactttgcggccgaggtccgcaagctgcgtaccttgttccagtctcttggttactacgattcttccaaggcatactacgccttcaaggtctcgttcaacctctgcatctggggtttgtcgacggtcattgtggccaagtggggccagacctcgaccctcgccaacgtgctctcggctgcgcttttgggtctgttctggcagcagtgcggatggttggctcacgactttttgcatcaccaggtcttccaggaccgtttctggggtgatcttttcggcgccttcttgggaggtgtctgccagggcttctcgtcctcgtggtggaaggacaagcacaacactcaccacgccgcccccaacgtccacggcgaggatcccgacattgacacccaccctctgttgacctggagtgagcatgcgttggagatgttctcggatgtcccagatgaggagctgacccgcatgtggtcgcgtttcatggtcctgaaccagacctggttttacttccccattctctcgtttgcccgtctctcctggtgcctccagtccattctctttgtgctgcctaacggtcaggcccacaagccctcgggcgcgcgtgtgcccatctcgttggtcgagcagctgtcgcttgcgatgcactggacctggtacctcgccaccatgttcctgttcatcaaggatcccgtcaacatgctggtgtactttttggtgtcgcaggcggtgtgcggaaacttgttggcgatcgtgttctcgctcaaccacaacggtatgcctgtgatctcgaaggaggaggcggtcgatatggatttcttcacgaagcagatcatcacgggtcgtgatgtccacccgggtctatttgccaactggttcacgggtggattgaactatcagatcgagcaccacttgttcccttcgatgcctcgccacaacttttcaaagatccagcctgctgtcgagaccctgtgcaaaaagtacaatgtccgataccacaccaccggtatgatcgagggaactgcagaggtctttagccgtctgaacgaggtctccaaggctgcctccaagatgggtaaggcgcagtaa

SEQ ID NO: 4 - protein, 457 aa
MAAAPSVRTFTRAEVLNAEALNEGKKDAEAPFLMIIDNKVYDVREFVPDHPGGSVILTHVGKDGTDVFDTFHPEAAWETLANFYVGDIDESDRDIKNDDFAAEVRKLRTLFQSLGYYDSSKAYYAFKVSFNLCIWGLSTVIVAKWGQTSTLANVLSAALLGLFWQQCGWLAHDFLHHQVFQDRFWGDLFGAFLGGVCQGFSSSWWKDKHNTHHAAPNVHGEDPDIDTHPLLTWSEHALEMFSDVPDEELTRMWSRFMVLNQTWFYFPILSFARLSWCLQSILFVLPNGQAHKPSGARVPISLVEQLSLAMHWTWYLATMFLFIKDPVNMLVYFLVSQAVCGNLLAIVFSLNHNGMPVISKEEAVDMDFFTKQIITGRDVHPGLFANWFTGGLNYQIEHHLFPSMPRHNFSKIQPAVETLCKKYNVRYHTTGMIEGTAEVFSRLNEVSKAASKMGKAQ

SEQ ID NO: 5 - DNA (other nucleic acid), 1200 bp; double-stranded, linear
  description: /desc = “DNA”
atggcacctcccaacactatcgatgccggtttgacccagcgtcatatcagcacctcggccccaaactcggccaagcctgccttcgagcgcaactaccagctccccgagttcaccatcaaggagatccgagagtgcatccctgcccactgctttgagcgctccggtctccgtggtctctgccacgttgccatcgatctgacttgggcgtcgctcttgttcctggctgcgacccagatcgacaagtttgagaatcccttgatccgctatttggcctggcctgtttactggatcatgcagggtattgtctgcaccggtgtctgggtgctggctcacgagtgtggtcatcagtccttctcgacctccaagaccctcaacaacacagttggttggatcttgcactcgatgctcttggtcccctaccactcctggagaatctcgcactcgaagcaccacaaggccactggccatatgaccaaggaccaggtctttgtgcccaagacccgctcccaggttggcttgcctcccaaggagaacgctgctgctgccgttcaggaggaggacatgtccgtgcacctggatgaggaggctcccattgtgactttgttctggatggtgatccagttcttgttcggatggcccgcgtacctgattatgaacgcctctggccaagactacggccgctggacctcgcacttccacacgtactcgcccatctttgagccccgcaactttttcgacattattatctcggacctcggtgtgttggctgccctcggtgccctgatctatgcctccatgcagttgtcgctcttgaccgtcaccaagtactatattgtcccctacctctttgtcaacttttggttggtcctgatcaccttcttgcagcacaccgatcccaagctgccccattaccgcgagggtgcctggaatttccagcgtggagctctttgcaccgttgaccgctcgtttggcaagttcttggaccatatgttccacggcattgtccacacccatgtggcccatcacttgttctcgcaaatgccgttctaccatgctgaggaagctacctatcatctcaagaaactgctgggagagtactatgtgtacgacccatccccgatcgtcgttgcggtctggaggtcgttccgtgagtgccgattcgtggaggatcagggagacgtggtctttttcaagaagtaa

SEQ ID NO: 6 - protein, 399 aa
MAPPNTIDAGLTQRHISTSAPNSAKPAFERNYQLPEFTIKEIRECIPAHCFERSGLRGLCHVAIDLTWASLLFLAATQIDKFENPLIRYLAWPVYWIMQGIVCTGVWVLAHECGHQSFSTSKTLNNTVGWILHSMLLVPYHSWRISHSKHHKATGHMTKDQVFVPKTRSQVGLPPKENAAAAVQEEDMSVHLDEEAPIVTLFWMVIQFLFGWPAYLIMNASGQDYGRWTSHFHTYSPIFEPRNFFDIIISDLGVLAALGALIYASMQLSLLTVTKYYIVPYLFVNFWLVLITFLQHTDPKLPHYREGAWNFQRGALCTVDRSFGKFLDHMFHGIVHTHVAHHLFSQMPFYHAEEATYHLKKLLGEYYVYDPSPIVVAVWRSFRECRFVEDQGDVVFFKK